SEQUENCE LISTING (10 sequences)

SEQ ID NO: 1 - protein, 513 aa, Streptomyces violaceusniger
LNNVVIVGGGTAGWMTASYLKAAFGDRIDITLVESGHIGAVGVGEATFSDIRHFFEFLGLKEKDWMPACNATYKLAVRFENWREKGHYFYHPFEQMRSVNGFPLTDWWLKQGPTDRFDKDCFVMASVIDAGLSPRHQDGTLIDQPFDEGADEMQGLTMSEHQGKTQFPYAYQFEAALLAKYLTKYSVERGVKHIVDDVREVSLDDRGWITGVRTGEHGDLTGDLFIDCTGFRGLLLNQALEEPFISYQDTLPNDSAVALQVPMDMERRGILPCTTATAQDAGWIWTIPLTGRVGTGYVYAKDYLSPEEAERTLREFVGPAAADVEANHIRMRIGRSRNSWVKNCVAIGLSSGFVEPLESTGIFFIHHAIEQLVKNFPAADWNSMHRDLYNSAVSHVMDGVREFLVLHYVAAKRNDTQYWRDTKTRKIPDSLAERIEKWKVQLPDSETVYPYYHGLPPYSYMCILLGMGGIELKPSPALALADGGAAQREFEQIRNKTQRLTEVLPKAYDYFTQ

SEQ ID NO: 2 - protein, 538 aa, Pseudomonas fluorescens
MNKPIKNIVIVGGGTAGWMAASYLVRALQQQVNITLIESAAIPRIGVGEATIPSLQKVFFDFLGIPEREWMPQVNGAFKAAIKFVNWRKPPDHSRDDYFYHLFGSVPNCDGVPLTHYWLRKREQGFQQPMEYACYPQPGALDGKLAPCLLDGTRQMSHAWHFDAHLVADFLKRWAVERGVNRVVDEVVEVRLNDRGYISTLLTKEGRTLEGDLFIDCSGMRGLLINQALKEPFIDMSDYLLCDSAVASAVPNDDVREGVEPYTSAIAMNSGWTWKIPMLGRFGSGYVFSSKFTSRDQATADFLNLWGLSDNQSLNQIKFRVGRNKRAWVNNCVSIGLSSCFLEPLESTGIYFIYAALYQLVKHFPDTSFDPRLSDAFNAEIVYMFDDCRDFVQAHYFTTSREDTPFWLANRHELRLSDAIKEKVQRYKAGLPLTTTSFDDSTYYETFDYEFKNFWLNGNYYCIFAGLGMLPDRSLPLLQHRPESIEKAEAMFASIRREAERLRTSLPTNYDYLRSLRNGDAGQSRNQRGPTLAAKEGL

SEQ ID NO: 3 - protein, 174 aa, Bacillus subtilis
MKVLVLAFHPNMEQSVVNRAFADTLKDAPGITLRDLYQEYPDEAIDVEKEQKLCEEHDRIVFQFPLYWYSSPPLLKKWLDHVLLYGWAYGTNGTALRGKEFMVAVSAGAPEEAYQAGGSNHYAISELLRPFQATSNFIGTTYLPPYVFYQAGTAGKSELAEGATQYREHVLKSF

SEQ ID NO: 4 - protein, 211 aa, Bacillus wakoensis
MTKVLYITAHPHDDTQSFSMAVGKAFIDTYKEVNPDHEVETIDLYIEDIPHIDVDVFSGWGKLRSGQGFDQLSSDEKAKVGRLSELCEQFVSADKYIFVSPLWNFSFPPVLKAYIDSVAVAGKTFKYTEQGPVGLLTDKKALHIQARGGIYSEGPAAQMEMGHRYLSIIMQFFGVPSFDGLFVEGHNAMPDKAQEIKEKAVARAKDLAHTF

SEQ ID NO: 5 - protein, 456 aa, Methylophaga aminisulfidivorans
MATRIAILGAGPSGMAQLRAFQSAQEKGAEIPELVCFEKQADWGGQWNYTWRTGLDENGEPVHSSMYRYLWSNGPKECLEFADYTFDEHFGKPIASYPPREVLWDYIKGRVEKAGVRKYIRFNTAVRHVEFNEDSQTFTVTVQDHTTDTIYSEEFDYVVCCTGHFSTPYVPEFEGFEKFGGRILHAHDFRDALEFKDKTVLLVGSSYSAEDIGSQCYKYGAKKLISCYRTAPMGYKWPENWDERPNLVRVDTENAYFADGSSEKVDAIILCTGYIHHFPFLNDDLRLVTNNRLWPLNLYKGVVWEDNPKFFYIGMQDQWYSFNMFDAQAWYARDVIMGRLPLPSKEEMKADSMAWREKELTLVTAEEMYTYQGDYIQNLIDMTDYPSFDIPATNKTFLEWKHHKKENIMTFRDHSYRSLMTGTMAPKHHTPWIDALDDSLEAYLSDKSEIPVAKEA

SEQ ID NO: 6 - protein, 456 aa, Artificial Sequence
  M15L/S23A mutant form of mMFO of Methylophaga aminisulfidivorans
MATRIAILGAGPSGLAQLRAFQAAQEKGAEIPELVCFEKQADWGGQWNYTWRTGLDENGEPVHSSMYRYLWSNGPKECLEFADYTFDEHFGKPIASYPPREVLWDYIKGRVEKAGVRKYIRFNTAVRHVEFNEDSQTFTVTVQDHTTDTIYSEEFDYVVCCTGHFSTPYVPEFEGFEKFGGRILHAHDFRDALEFKDKTVLLVGSSYSAEDIGSQCYKYGAKKLISCYRTAPMGYKWPENWDERPNLVRVDTENAYFADGSSEKVDAIILCTGYIHHFPFLNDDLRLVTNNRLWPLNLYKGVVWEDNPKFFYIGMQDQWYSFNMFDAQAWYARDVIMGRLPLPSKEEMKADSMAWREKELTLVTAEEMYTYQGDYIQNLIDMTDYPSFDIPATNKTFLEWKHHKKENIMTFRDHSYRSLMTGTMAPKHHTPWIDALDDSLEAYLSDKSEIPVAKEA

SEQ ID NO: 7 - protein, 456 aa, Artificial Sequence
  M15L/S23A/C78I mutant form of mMFO of Methylophaga aminisulfidivorans
MATRIAILGAGPSGLAQLRAFQAAQEKGAEIPELVCFEKQADWGGQWNYTWRTGLDENGEPVHSSMYRYLWSNGPKEILEFADYTFDEHFGKPIASYPPREVLWDYIKGRVEKAGVRKYIRFNTAVRHVEFNEDSQTFTVTVQDHTTDTIYSEEFDYVVCCTGHFSTPYVPEFEGFEKFGGRILHAHDFRDALEFKDKTVLLVGSSYSAEDIGSQCYKYGAKKLISCYRTAPMGYKWPENWDERPNLVRVDTENAYFADGSSEKVDAIILCTGYIHHFPFLNDDLRLVTNNRLWPLNLYKGVVWEDNPKFFYIGMQDQWYSFNMFDAQAWYARDVIMGRLPLPSKEEMKADSMAWREKELTLVTAEEMYTYQGDYIQNLIDMTDYPSFDIPATNKTFLEWKHHKKENIMTFRDHSYRSLMTGTMAPKHHTPWIDALDDSLEAYLSDKSEIPVAKEA

SEQ ID NO: 8 - protein, 456 aa, Artificial Sequence
  M15L/S23A/Y207W mutant form of mMFO of Methylophaga aminisulfidivorans
MATRIAILGAGPSGLAQLRAFQAAQEKGAEIPELVCFEKQADWGGQWNYTWRTGLDENGEPVHSSMYRYLWSNGPKECLEFADYTFDEHFGKPIASYPPREVLWDYIKGRVEKAGVRKYIRFNTAVRHVEFNEDSQTFTVTVQDHTTDTIYSEEFDYVVCCTGHFSTPYVPEFEGFEKFGGRILHAHDFRDALEFKDKTVLLVGSSWSAEDIGSQCYKYGAKKLISCYRTAPMGYKWPENWDERPNLVRVDTENAYFADGSSEKVDAIILCTGYIHHFPFLNDDLRLVTNNRLWPLNLYKGVVWEDNPKFFYIGMQDQWYSFNMFDAQAWYARDVIMGRLPLPSKEEMKADSMAWREKELTLVTAEEMYTYQGDYIQNLIDMTDYPSFDIPATNKTFLEWKHHKKENIMTFRDHSYRSLMTGTMAPKHHTPWIDALDDSLEAYLSDKSEIPVAKEA

SEQ ID NO: 9 - protein, 713 aa, Artificial Sequence
  tryptophan halogenase-flavin reductase fusion enzyme (Thal-FRE)
MGSSHHHHHHSSGLVPRGSHLNNVVIVGGGTAGWMTASYLKAAFGDRIDITLVESGHIGAVGVGEATFSDIRHFFEFLGLKEKDWMPACNATYKLAVRFENWREKGHYFYHPFEQMRSVNGFPLTDWWLKQGPTDRFDKDCFVMASVIDAGLSPRHQDGTLIDQPFDEGADEMQGLTMSEHQGKTQFPYAYQFEAALLAKYLTKYSVERGVKHIVDDVREVSLDDRGWITGVRTGEHGDLTGDLFIDCTGFRGLLLNQALEEPFISYQDTLPNDSAVALQVPMDMERRGILPCTTATAQDAGWIWTIPLTGRVGTGYVYAKDYLSPEEAERTLREFVGPAAADVEANHIRMRIGRSRNSWVKNCVAIGLSSGFVEPLESTGIFFIHHAIEQLVKNFPAADWNSMHRDLYNSAVSHVMDGVREFLVLHYVAAKRNDTQYWRDTKTRKIPDSLAERIEKWKVQLPDSETVYPYYHGLPPYSYMCILLGMGGIELKPSPALALADGGAAQREFEQIRNKTQRLTEVLPKAYDYFTQSGSAAGMKVLVLAFHPNMEQSVVNRAFADTLKDAPGITLRDLYQEYPDEAIDVEKEQKLCEEHDRIVFQFPLYWYSSPPLLKKWLDHVLLYGWAYGTNGTALRGKEFMVAVSAGAPEEAYQAGGSNHYAISELLRPFQATSNFIGTTYLPPYVFYQAGTAGKSELAEGATQYREHVLKSF

SEQ ID NO: 10 - protein, 573 aa, Artificial Sequence
  PTDH-AzoA fusion enzyme
MGSSHHHHHHSSGLVPRGSHMLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKANPAADSRSAAGMTKVLYITAHPHDDTQSFSMAVGKAFIDTYKEVNPDHEVETIDLYIEDIPHIDVDVFSGWGKLRSGQGFDQLSSDEKAKVGRLSELCEQFVSADKYIFVSPLWNFSFPPVLKAYIDSVAVAGKTFKYTEQGPVGLLTDKKALHIQARGGIYSEGPAAQMEMGHRYLSIIMQFFGVPSFDGLFVEGHNAMPDKAQEIKEKAVARAKDLAHTF

(19) United States
(12) Patent Application Publication
(10) Pub. No.: US 2022/0267948 A1
(43) Pub. Date: Aug. 25, 2022

(54) PROCESS FOR DYEING TEXTILES

(71) Applicants: Sanko Tekstil Isletmeleri San. Ve Tic. A.S., Inegol - BURSA (TR); RIJKSUNIVERSITEIT GRONINGEN, Groningen (NL)

(72) Inventors: Nikola LONCAR, Groningen (NL); Fatih KONUKOGLU, Inegol - BURSA (TR); Marco W. FRAAIJE, Groningen (NL); Jitka ERYILMAZ, Inegol - BURSA (TR); Esref TUNCER, Inegol - BURSA (TR)

(21) Appl. No.: 17/627,156

(22) PCT Filed: Oct. 31, 2019
(86) PCT No.: PCT/EP2019/079911

§ 371 Date: Jan. 14, 2022

(30) Foreign Application Priority Data
Jul. 22, 2019 (EP) PCT/EP2019/069720

(51) Int. Cl. D06P 1/22; C09B 67/30; C09B 7/00; D06P 5/02; D06P 1/00; D06P 1/46

(52) U.S. Cl. CPC D06P 1/228; C09B 67/0078; C09B 7/00; D06P 5/02; D06P 1/008; D06P 1/46; D06P 1/002; C12Y 401/99001; C12Y 113/11052; C12Y 114/19; C12Y 114/14

(57) ABSTRACT

The present invention relates to a process for dyeing textiles, in particular for dyeing textiles using enzymes. The present invention also relates to a method for producing leuco indigo and/or leuco forms of indigo derivatives. The present invention further refers to textiles obtainable through said process, to an apparatus comprising a reactor containing enzymes, and to a microbial flavin-containing monooxygenase.

### FIELD OF THE INVENTION

The present invention relates to a process for dyeing textiles, in particular for dyeing textiles using enzymes. The present invention also relates to a method for producing leuco indigo and/or indigo and/or derivatives thereof.

### BACKGROUND OF THE INVENTION

Vat dyes are insoluble dyes that require a reducing agent to be solubilized in water. Conventionally, dyeing with vat dyes includes applying the dye in its soluble, reduced form to the textiles and subsequently oxidizing the dye back to the insoluble form, which confers color to the textile.

Indigo is a vat dye of Formula I:

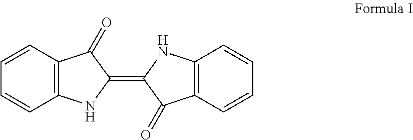 [SMILES: O=C1/C(=C2\Nc3ccccc3C2=O)Nc2ccccc21]

Substitutions on the indigo aromatic ring(s) with groups such as halogen, alkyl, alkoxy, amino, aryl, aryloxy, and carbonyl, provide compounds that span in a wide range of colors other than blue, and are part of the so-called indigo derivatives.

Indigo, as well as its derivatives, is typically reduced to its leuco form (i.e., to leuco indigo), which is water soluble, in order to be applied to a textile to be dyed. Leuco indigo (also known as white indigo) is thus the reduced, water soluble, form of indigo. In currently available industrial dyeing processes indigo is treated with reducing agents to obtain an aqueous solution comprising leuco-indigo, which is, subsequently, applied to textiles. Indigo is then obtained by oxidation of leuco-indigo on the textile. Oxidation of leuco indigo to indigo can be carried out, for example, by exposing the textile treated with leuco indigo to air, so that leuco indigo is oxidized by reaction with the oxygen in the air.

Currently available dyeing processes have several drawbacks.

As above mentioned, to obtain leuco indigo, indigo is treated with reducing chemical agents. Currently available reducing chemical agents are, usually, harsh chemicals, i.e. hazardous chemicals for users and/or environment, such as sodium hydroxide and sodium hydrosulfite.

Moreover, fabrics and textiles in general may be damaged by long time and/or repeated exposure to highly alkaline conditions.

Additionally, large quantities of reducing salts and hydroxides are used in conventional dyeing processes, thus generating great amounts of wastewater that must be treated before being disposed, thus increasing costs of the dyeing process.

### SUMMARY OF THE INVENTION

It is an aim of the present invention to solve the above mentioned problems and to provide a process for dyeing textiles that is safe, cost-effective and environmentally friendly.

Another aim of the present invention is to provide a process for dyeing textiles which is fast, effective and easy to carry out.

Also aim of the present invention is to provide a process for dyeing textiles that is sustainable with respect to conventional processes.

The above aims, as well as others, are reached through the present invention that provides a process according to claim 1, namely a process for dyeing textiles comprising at least two enzymatic reactions to enzymatically produce leuco indigo and/or the leuco form of an indigo derivative.

The present invention also relates to a dyed textile according to claim 16, i.e., a dyed textile article as obtainable according to the process of the invention, to a method for the production of leuco indigo or the leuco form of an indigo derivative, by enzymatic synthesis, according to claim 17; to an apparatus according to claim 21; and to a monooxygenase enzyme according to claim 23.

Preferred embodiments of the invention are object of dependent claims 2 to 14, 18 to 20, and 22.

### BRIEF DESCRIPTION OF THE FIGURES

FIG. 1 is a schematic representation of an embodiment of the process of the invention;

FIG. 2 is a schematic representation of another embodiment of the process of the invention;

### DETAILED DESCRIPTION

The present invention relates to a process for dyeing textiles comprising the following steps:
a) hydroxylating indole or an indole derivative in the presence of at least an oxidizing enzyme, to obtain indoxyl or an indoxyl derivative;
b) converting said indoxyl or said indoxyl derivative to leuco indigo or to a leuco form of an indigo derivative in the presence of at least a reducing enzyme;
c) providing at least said leuco indigo or said leuco form of said indigo derivative to at least part of a textile; and
d) oxidizing at least part of said leuco indigo or said leuco form of said indigo derivative, so that indigo or an indigo derivative is produced on said textile, to dye at least part of said textile.

It has been surprisingly found that, through the process of the invention, it is possible to dye textiles avoiding, or substantially avoiding, the use of harsh chemicals. Moreover, it has been surprisingly found that, through the process of the invention it is possible to produce insoluble dyes, such as indigo, on a textile while avoiding or substantially avoiding the precipitation of insoluble dyes in the reaction mixture, i.e., in the mixture containing the enzymes.

In particular, it has been observed that, starting from indole, or an indole derivative, using at least an oxidizing enzyme and at least a reducing enzyme, leuco indigo or a leuco forms of indigo derivatives, may be obtained in a fast and effective way, substantially avoiding or avoiding the precipitation of insoluble dyes in the reaction mixture. Without being bound to a specific scientific explanation, the process may involve dimerization of indoxyl to indigo and immediate reduction of indigo to its leuco form by the reducing enzyme. In addition to the wild type reductases, suitably genetically modified reductases may be engineered to reduce indigo before it precipitates in the reactor.

Advantageously, without being bound to a specific scientific explanation, it has been observed that, starting from indole or derivatives thereof, through the process of the invention, it is possible to produce several different dyes, as well as the leuco form thereof, thus avoiding the use of harsh chemicals, such as, for example, sodium hydrosulfite, sodium hydroxide, and solvents.

According to an aspect, the process of the present invention allows the production of dyed textiles. Dyed textiles obtainable through the process of the invention may have a variety of colors. In fact, advantageously, by varying the reagents (e.g., indole or a derivative thereof) in the process of the invention, different dyes, as well as leuco forms thereof, may be obtained, through enzymatic reactions, so that different final colors can be imparted to textiles.

Also advantageously, reagents suitable to be used in the process of the invention have a low cost, so that the process of the invention results to be particularly cost-effective with respect to the currently available dyeing processes.

According to an aspect, as above mentioned, the process of the invention includes a step of hydroxylating indole or an indole derivative in the presence of at least an oxidizing enzyme, to obtain indoxyl or an indoxyl derivative. Indoxyl and indoxyl derivatives are subsequently converted to leuco indigo and leuco forms of indigo derivatives, respectively. As above discussed, without being bound to a specific scientific explanation, the process may involve dimerization of indoxyl to indigo and immediate reduction of indigo to its leuco form by the reducing enzyme.

As used therein, the term “leuco indigo” refers to the reduced form of indigo. According to the present description, the term “leuco indigo” encompasses leuco indigo in the forms present in the reaction mixtures and as present in aqueous solutions including leuco indigo for textile dyeing. Such reaction mixtures and aqueous solution may include leuco indigo in any suitable concentration; in particular the concentration of leuco indigo in the reaction mixtures and in solutions that are to be stocked is high and is typically greater than the concentration of leuco indigo in reaction mixtures and in aqueous solutions suitable for dyeing a textile.

As used herein, the terms “indole derivatives”, “indoxyl derivatives”, “indigo derivatives”, “indigo derivatives” and “leuco form of indigo derivatives”, refer to respectively indole, indoxyl, indigo and leuco form of indigo substituted with one or more substituents, for example substituted with: one or more groups on one or more carbons in any position selected from positions 4, 5, 6 and 7 of indole or indoxyl, and from positions 4, 4′, 5, 5′, 6, 6′, 7, and 7′ of indigo, and/or by a group on the nitrogen atom(s) of indole, indoxyl or indigo. The one or more groups substituting one or more carbons may be groups such as, but not limited to, halogen groups, alkyl groups, alkoxy groups, aryl groups, aryloxy groups, amine groups, nitro groups and carbonyl groups. The group substituting nitrogen atom(s) may be groups such as, but not limited to, alkyl groups, aryl groups, and acyl groups. Therefore, indole derivatives may be, for example, 4-chloroindole, 5-chloroindole, 6-chloroindole, 7-chloroindole, 5-bromoindole, 6-bromoindole, 5-nitroindole, 5-hydroxyindole, 5-methylindole, 5-methoxyindole, 6-methylindole, 7-methylindole, 5-aminoindole, 1-methylindole, indole-6-carboxaldehyde; and indoxyl derivatives can be, for example, 4-chloroindoxyl, 5-chloroindoxyl, 6-chloroindoxyl, 7-chloroindoxyl, 5-bromoindoxyl, 6-bromoindoxyl, 5-nitroindoxyl, 5-hydroxyindoxyl, 5-methylindoxyl, 5-methoxyindoxyl, 6-methylindoxyl, 7-methylindoxyl, 5-aminoindoxyl, 1-methylindoxyl, indoxyl-6-carboxaldehyde. Any other indole and indoxyl derivatives may be used in the process of the invention, provided that such indole derivatives can be reacted and converted into the correspondent indoxyl derivatives by enzymatic oxidation. These indoxyl derivatives, when dimerized, provide the correspondent indigo derivatives, which have each a different color. As used herein, the term “indigo derivatives” refer also to asymmetric indigo, i.e. indigo deriving from dimerization of two different indoxyl derivatives, or of indoxyl and an indoxyl derivative. Dyeing of the textile with asymmetric indigo can be achieved according to the process of the invention when two or more different indole derivatives, or indole and one or more indole derivatives, are used. For example, when two different indole derivatives, or indole and an indole derivative, are used, two different indoxyl derivatives, or indoxyl and an indoxyl derivative, are obtained. Advantageously, when such two different indoxyl derivatives, or indoxyl and an indoxyl derivative, are used, three different indigo derivatives are obtained (namely, two different symmetric indigo derivatives and an asymmetric indigo derivative), so that a textile can be dyed with more than one dye, in particular, by providing the leuco form of such indigo derivatives to the textile, and oxidizing said derivatives to produce the dye onto the textile.

According to embodiments, the indole derivative is 6-bromoindole, and said indigo derivative is Tyrian purple.

As used herein, the term “oxidizing enzyme” refers to any enzyme that is able to catalyze oxidation of its substrates. Oxidizing enzymes that are suitable to be used in the process of the invention are known in the art. Suitable enzymes are monooxygenases, preferably flavin-containing monooxygenases (FMOs), and more preferably microbial flavin-containing monooxygenases (mFMOs). For example, a suitable monooxygenase is mFMO of Methylophaga aminisulfidivorans. Another suitable monooxygenase is FMO of Nitrincola lacisaponensis (NiFMO). Alternatively, the monooxygenase can be a Baeyer-Villiger monooxygenase (BVMO). Monooxygenases, in particular FMOs and mFMOs, provide good conversion rates and of indole and derivatives thereof, and are thus suitable to be used in the process of the invention. Baeyer-Villiger monooxygenases (BVMOs) have close homology to FMOs, and are thus suitable as well to be used in the process of the invention. As used herein, the term “oxidizing enzyme” also encompasses genetically modified oxidizing enzymes, e.g. oxidizing enzymes that have been genetically modified to improve the enzyme's properties, such as oxidation efficiency of the substrate(s) of the oxidizing enzyme.

Without being bound to a specific scientific explanation, it has been observed that oxidizing enzymes, suitable to be used in the process of the invention, catalyze the hydroxylation of indole and/or indole derivative(s), to provide indoxyl and/or the corresponding indoxyl derivative(s).

Indoxyl and indoxyl derivatives dimerize to indigo, and indigo derivatives, respectively. In other words, conversion of indoxyl (or indoxyl derivatives) into indigo (or indigo derivatives) occurs spontaneously, by dimerization.

As above mentioned, according to the process of the invention, indoxyl and/or indoxyl derivatives, in the presence of at least a reducing enzyme, are converted to obtain leuco indigo or the leuco form of an indigo derivative. Without being bound to a specific scientific explanation, the process may involve dimerization of indoxyl to indigo and immediate reduction of indigo to its leuco form by the reducing enzyme.

As used herein, the term “reducing enzyme” refers to any enzyme that is able to catalyze reduction of its substrates. Reducing enzymes that are suitable to be used in the process of the invention are known in the art. Suitable enzymes are reductases, preferably azoreductases, more preferably flavin-dependent azoreductases. For example, a NADH- and flavin-dependent azoreductase suitable to be used in the process of the invention is AzoA, from Bacillus sp, which is an enzyme that is known per se, from Suzuki et al., “Azoreductase from alkaliphilic Bacillus sp. AO1 catalyzes indigo reduction”, Applied Microbiology and Biotechnology (2018) 102:9171-9181. For example, a suitable reducing enzyme is the AzoA reductase of Bacillus wakoensis having sequence MTKVLYITAHPHDDTQSFSMAVGKAFIDTYKEVNPDHEVETIDLYIEDIPHID VDVFSGWGKLRSGQGFDQLSSDEKAKVGRLSELCEQFVSADKYIFVSPLWN FSFPPVLKAYIDSVAVAGKTFKYTEQGPVGLLTDKKALHIQARGGIYSEGPA AQMEMGHRYLSIIMQFFGVPSFDGLFVEGHNAMPDKAQEIKEKAVARAKDL AHTF (SEQ. ID NO. 4). According to embodiments, a suitable reducing enzyme may have a sequence having sequence identity of at least 80% with respect to SEQ. ID NO. 4. As used herein, the term “reducing enzyme” also encompasses genetically modified reducing enzymes, e.g. reducing enzymes that have been genetically modified to improve the enzyme's properties, such as reduction efficiency of the substrate(s) of the reducing enzyme.

As above mentioned, it has been observed that, starting from indoxyl and/or indoxyl derivatives, in the presence of at least a reducing enzyme, leuco indigo or the leuco form of indigo derivatives can be obtained.

According to an aspect, the process of the invention includes a step of providing at least the leuco indigo or a leuco form of an indigo derivative to at least part of a textile, wherein the leuco indigo and the leuco form of an indigo derivative are enzymatically obtained, i.e., are obtained through enzymatic reaction. After that leuco indigo (or the leuco form of an indigo derivative) is provided to at least part of a textile, leuco indigo (or the leuco form of an indigo derivative) is oxidized, so that indigo or an indigo derivative is produced on the textile, to obtain a textile that is at least in part dyed. Oxidation of leuco indigo and/or leuco forms of indigo derivatives may be carried out according to known methods. For example, a textile may be impregnated with a solution including leuco indigo (or a leuco form of a indigo derivative), and subsequently exposed to air. Such exposition to air allows for the oxidation of leuco indigo into indigo. Such oxidation occurs on the textile, thus resulting in dyeing of the textile.

As used herein, terms “textile”, “textiles” and “textile article(s)” refer to any fibers, yarns, ropes, fabrics and/or garments able to be dyed, for example by indigo and/or derivatives thereof. In embodiments, the textile material may include natural fibers, such as fibers deriving from animals or plants, e.g. cotton, linen, silk, wool fibers, and mixtures thereof. In embodiments, the textile materials may include synthetic fibers, such as, for example, polyester, rayon, nylon, lycra and mixtures thereof. In embodiments, the textile may include mixtures of natural and synthetic fibers. For example, suitable textiles may be elasticized cotton fabrics or garments. In embodiments, in the textile may include regenerated fibers or yarns, in addition to or as an alternative to natural and/or synthetic fibers and yarns. In the present description, regenerated yarns are yarns that include regenerated fibers. Regenerated fibers, or man made fibers, are commercially available. For example, suitable regenerated fibers can be selected from rayon, lyocell, modal, viscose, bamboo, and mixture thereof. Moreover, said yarns may be manufactured by any known method, and said fabrics also may be manufactured by any known method, such as weaving, knitting, crocheting, knotting, and felting. Furthermore, said garments may be any garment, such as jeans, shirts, casual wear garments, etc.

According to embodiments, the process of the invention further comprises a step of converting tryptophan or a tryptophan derivative in the presence of at least a tryptophanase, to obtain the indole or the indole derivative. In other words, tryptophan and/or a tryptophan derivative can be used as starting material (i.e., starting substrate) in the process of the invention, to enzymatically produce indole or indole derivatives. Accordingly, tryptophan and/or a tryptophan derivative can be used as starting materials (i.e., starting substrates) to obtain leuco indigo and/or a leuco form of an indigo derivative, through a plurality of enzymatic reactions.

Tryptophanases (systematic name: L-tryptophan indole-lyase (deaminating; pyruvate-forming)) are enzymes, per se known, that cleave a carbon-carbon bond of tryptophan, releasing indole. They may use pyridoxal phosphate (PLP) as cofactor. According to embodiments of the invention, PLP can be optionally used to improve the yield of the enzymatic conversion of tryptophan or of its derivatives catalyzed by tryptophanase. Tryptophanases suitable to be used in the process of the invention are known in the art. For example, a tryptophanase suitable to be used in the method of the invention is the tryptophanase of Escherichia coli NEB® 10β.

As used herein, the term “tryptophan derivative” refers to tryptophan substituted with one or more substituents, as above disclosed, mutatis mutandis, with reference to indole, indoxyl, indigo and leuco-indigo derivatives. For example, a tryptophan derivative may be a halogenated derivative of tryptophan, i.e., halogenated tryptophan (e.g., 6-bromotryptophan).

According to embodiments, the tryptophan derivative is halogenated tryptophan, and the process of the invention further comprises a step of halogenating tryptophan, in the presence of at least a tryptophan halogenase and a halogen source, to obtain said halogenated tryptophan.

According to embodiments, the tryptophan derivative is a 6-bromotryptophan (i.e., a halogenated tryptophan) and the indigo derivative is Tyrian purtple.

Tryptophan halogenases are enzymes, that are per se known, able to catalyze the halogenation of tryptophan in various positions. Tryptophan halogenases are usually flavin-dependent halogenases, i.e. they usually use FAD or FADH₂ as a cofactor. Tryptophan halogenases suitable to be used in the process of the invention are known in the art. For example, tryptophan halogenases suitable to be used in the process of the invention are tryptophan halogenases, such as, for example the tryptophan halogenase of Streptomyces violaceusniger.

According to embodiments, the tryptophan halogenase is the tryptophan halogenase of the strain SPC6 of Streptomyces violaceusniger.

For example, the tryptophan halogenase may have the following sequence:

(SEQ. ID NO. 1)
LNNVVIVGGGTAGWMTASYLKAAFGDRIDITLVESGHIGAVGVGEATFSD
IRHFFEFLGLKEKDWMPACNATYKLAVRFENWREKGHYFYHPFEQMRSVN
GFPLTDWWLKQGPTDRFDKDCFVMASVIDAGLSPRHQDGTLIDQPFDEGA
DEMQGLTMSEHQGKTQFPYAYQFEAALLAKYLTKYSVERGVKHIVDDVRE
VSLDDRGWITGVRTGEHGDLTGDLFIDCTGFRGLLLNQALEEPFISYQDT
LPNDSAVALQVPMDMERRGILPCTTATAQDAGWIWTIPLTGRVGTGYVYA
KDYLSPEEAERTLREFVGPAAADVEANHIRMRIGRSRNSWVKNCVAIGLS
SGFVEPLESTGIFFIHHAIEQLVKNFPAADWNSMHRDLYNSAVSHVMDGV
REFLVLHYVAAKRNDTQYWRDTKTRKIPDSLAERIEKWKVQLPDSETVYP
YYHGLPPYSYMCILLGMGGIELKPSPALALADGGAAQREFEQIRNKTQRL
TEVLPKAYDYFTQ.

This type of tryptophan halogenase catalyzes preferably the halogenation on the carbon in position 6 of tryptophan, whereby it is suitable to produce Tyrian purple (6,6′-dibromoindigo) according to the method of the invention.

According to embodiments, a suitable tryptophan halogenase may have a sequence having sequence identity of at least 80% with respect to SEQ. ID NO. 1.

Another tryptophan halogenase suitable for the method of the invention is tryptophan halogenase PrnA, preferably is the PrnA of Pseudomonas fluorescens, which catalyzes preferably the halogenation of tryptophan on the carbon in position 5 or 7 of the tryptophan.

For example, the tryptophan halogenase (PrnA) may have the following sequence:

(SEQ. ID. NO. 2)
MNKPIKNIVIVGGGTAGWMAASYLVRALQQQVNITLIESAAIPRIGVGEA
TIPSLQKVFFDFLGIPEREWMPQVNGAFKAAIKFVNWRKPPDHSRDDYFY
HLFGSVPNCDGVPLTHYWLRKREQGFQQPMEYACYPQPGALDGKLAPCLL
DGTRQMSHAWHFDAHLVADFLKRWAVERGVNRVVDEVVEVRLNDRGYIST
LLTKEGRTLEGDLFIDCSGMRGLLINQALKEPFIDMSDYLLCDSAVASAV
PNDDVREGVEPYTSAIAMNSGWTWKIPMLGRFGSGYVFSSKFTSRDQATA
DFLNLWGLSDNQSLNQIKFRVGRNKRAWVNNCVSIGLSSCFLEPLESTGI
YFIYAALYQLVKHFPDTSFDPRLSDAFNAEIVYMFDDCRDFVQAHYFTTS
REDTPFWLANRHELRLSDAIKEKVQRYKAGLPLTTTSFDDSTYYETFDYE
FKNFWLNGNYYCIFAGLGMLPDRSLPLLQHRPESIEKAEAMFASIRREAE
RLRTSLPTNYDYLRSLRNGDAGQSRNQRGPTLAAKEGL.

According to embodiments, a suitable tryptophan halogenase may have a sequence having sequence identity of at least 80% with respect to SEQ. ID NO. 2.

According to embodiments, the tryptophan halogenase may be a genetically modified enzyme; in other words, the tryptophan halogenase may be in a mutant form. For example, the tryptophan halogenase may be a mutant form of the tryptophan halogenase of the strain SPC6 of Streptomyces violaceusniger, or a mutant form of the tryptophan halogenase PrnA.

As used herein, the term “halogenated derivative” refers to any tryptophan, indole, indoxyl and indigo substituted with a halogen atom, in particular fluorine, chlorine, bromine or iodine atom, on one or more carbons in position 5, 6, 7 and 8 (and also 5′, 6′, 7′ and 8′ for indigo). For example, halogenated derivatives of tryptophan may be 6-bromotryptophan and 7-chlorotryptophan, halogenated derivatives of indole may be 6-bromoindole and 7-chloroindole, halogenated derivatives of indoxyl may be 6-bromoindoxyl and 7-chloroindoxyl, and halogenated derivatives of indigo may be Tyrian purple (i.e., 6,6′-dibromoindigo) and 7,7′-dichloroindigo.

Tryptophan halogenases convert tryptophan to a halogenated derivative of tryptophan, i.e., halogenated tryptophan, in presence of a halogen source. Halogen sources suitable to be used in the process of the invention are, for example, halogen salts, i.e. salts wherein the anion is halide ion. Suitable halogen salts are, for example, magnesium, silver, sodium, potassium, lithium, and calcium halogen salts, for example NaCl, KCl, KI, LiCl, CuCl₂, CuBr₂, AgCl, CaCl₂, CaBr₂, ClF, MgCl₂, MgBr₂, KBr, etc.

According to embodiments, the enzymatic production of leuco indigo and/or of the leuco form of an indigo derivative is carried out in a single reactor, as a one-pot process.

In other words, according to embodiments, at least the step of hydroxylating indole or an indole derivative in the presence of at least an oxidizing enzyme, to obtain indoxyl or an indoxyl derivative, and said step of converting said indoxyl or said indoxyl derivative to leuco indigo or to a leuco form of an indigo derivative in the presence of at least a reducing enzyme, are carried out as a one-pot process.

As used herein, the term “one-pot process” refers to a process wherein one or more reactants are subjected to successive enzymatic and/or non-enzymatic reactions in the same reactor. Advantageously, one-pot processes allow to substantially avoid or avoid separation and purification processes of the intermediate compounds, thus saving time and resources while increasing the overall yield of the process.

According to embodiments, the process of the invention may be carried out as a one-pot process by providing, in the same reactor, a mixture, particularly an aqueous mixture, including, for example, indole (or an indole derivative), an oxidizing enzyme, a reducing enzyme, and suitable cofactors, if required. In this case, indole (or the indole derivative) is hydroxylated by the oxidizing enzyme, thus obtaining indoxyl (or an indoxyl derivative) which, in presence of the reducing enzyme, is converted to leuco indigo (or to the leuco form of the indigo derivative deriving from the indoxyl derivative).

According to embodiments, leuco indigo (or the leuco form of an indigo derivative) can be provided to a textile, for example, by dipping the textile in the reactor containing the leuco indigo, i.e., in the reactor wherein the enzymatic conversion of indole into leuco indigo occurred.

Advantageously, the process for dyeing textiles of the invention, may be carried out in an aqueous medium. A textile may be dipped in the reactor containing leuco indigo, as well as the enzymes used to produce said leuco indigo, to be impregnated with the leuco indigo solution.

According to an aspect, the process of the invention involves a plurality of enzymatic reactions, that are preferably carried out in aqueous medium as a one-pot process. Advantageously, condition of the process such as, for example, temperature, pH, duration may be adjusted according to the enzymes and reagents used.

According to embodiments, the process of the invention may be carried out as a one-pot process by providing, in the same reactor, for example, tryptophan, a tryptophanase, an oxidizing enzyme, a reducing enzyme, and suitable cofactors, if required. In this case, tryptophan is enzymatically converted into indole by the tryptophanase. The enzymatic reactions leading to leuco indigo from indole occur as above discussed.

According to embodiments, the process of the invention may be carried out as a one-pot process by providing, in the same reactor, for example, tryptophan, a tryptophan halogenase, a halogen source, a tryptophanase, an oxidizing enzyme, a reducing enzyme, and suitable cofactors, if required. In this case, tryptophan is enzymatically halogenated by the tryptophan halogenase, to obtain a halogenated tryptophan. Said halogenated tryptophan is converted into the correspondent indole derivative by the tryptophanase. The halogenated derivative of indole is hydroxylated by the oxidizing enzyme to obtain the corresponding indoxyl derivative, which is converted to the leuco form of the corresponding halogenated derivative of indigo in presence of the reducing enzyme.

According to an aspect, the present invention relates to an apparatus for carrying out the process of the invention, comprising at least a reactor containing enzymes, wherein said enzymes include an oxidizing enzyme, preferably a monooxygenase, and a reducing enzyme, preferably an azoreductase, preferably also a tryptophanase, and optionally also a tryptophan halogenase.

Advantageously, the apparatus of the invention allows for the production of leuco indigo or leuco froms of indigo derivatives, starting from indole (or indole derivatives) or, preferably, from tryptophan or a tryptophan derivative.

Indigo or indigo derivatives may be obtained from leuco indigo (or leuco form of indigo derivatives) according to standard techniques, such as, for example, standard exposure to air. Advantageously, when a textile is provided with leuco indigo and exposed to air, oxygen in the air oxidizes such leuco indigo to indigo on the surface of the textile.

According to embodiments of the invention, the reaction mixture, e.g. an aqueous mixture, including the enzymes, may comprise other functional solutes, such as salts, buffering agents, and oxygen and/or peroxide scavengers (e.g. catalases). Catalase may be included in the reaction mixture to convert possibly formed H₂O₂ into O₂ and H₂O. For example, an exemplary reaction mixture may include, a suitable buffer, indole, a monooxygenase, a reductase, one or more cofactors, one or more cofactor regenerating enzymes and optionally a catalase. Preferably, the monooxygenase and the reductase are provided as fusion enzymes, i.e., as enzymes fused with a cofactor regenerating enzyme, such as for example, PTDH-mFMO and PTDH-AzoA.

According to embodiments, the enzymes used in the process of the invention are isolated enzymes. In other words, according to embodiments, the oxidizing enzyme, and/or the reducing enzyme, and/or the tryptophanase and/or the tryptophan halogenase used in the process of the invention are isolated from the host cell (e.g., bacterial cells, such as, for example, E. coli) in which the enzymes are produced. Enzymes may be isolated and/or purified from host cells and organisms according to techniques that are known in the art.

According to embodiments, one or more of the enzymes used in the process of the invention are immobilized enzymes. In other words, according to embodiments, the oxidizing enzyme, and/or the reducing enzyme, and/or the tryptophanase and/or the tryptophan halogenase used in the process of the invention are immobilized enzymes. As used herein, the term “immobilized enzyme” refers to enzymes that are bound, preferably covalently bound, to carriers, for example to epoxy-activated resins (such as methacrylate copolymers, e.g. Eupergit®, SepaBeads®, Relizyme™, Purolite®), cellulose, agarose, polystyrenic ion exchange resins, amino acrylate resins, hydrogels (immobilization by occlusion; e.g. agarose, alginate, carrageenan or gelatin), chelating carriers (e.g. Ni-Sepharose®, IDA-Sepharose®, NTA-Sepharose®, IDA-Agarose and derivatives of), etc. The type of carriers used to immobilize enzymes might depend on which are the exposed groups of the enzymes. For example, if surface amino groups are exposed on the enzymes, epoxy-activated resins may be used as carriers: as the amino groups covalently attaches to the epoxy groups of the epoxy-activated resins, the enzymes are immobilized onto the epoxy-activated resins.

Immobilization of enzymes may be performed according to techniques that are known in the art.

It has been observed that, advantageously, when the enzymes are immobilized, a high catalytic efficiency is retained, even after repeated catalytic cycles, i.e., after a prolonged use of the enzymes in the process of the invention.

According to embodiments, when the enzymes are immobilized, enzymes may be arranged, for example, within the reaction pot (i.e., the reactor), according to a sequence so that the reaction product of an enzyme is the substrate for the subsequent enzyme. In this case, advantageously, a flow in the reactor may be generated, so that the selected starting material (e.g., indole) is converted into leuco indigo (or the leuco form of an indigo derivative) flowing from an enzyme to another.

According to embodiments, the oxidizing enzyme is an oxygenase, preferably a monooxygenase, more preferably a flavin-containing monooxygenase (FMO), even more preferably a microbial flavin-containing monooxygenase (mFMO).

According to embodiments, the reducing enzyme is a reductase, preferably an azoreductase, more preferably a flavin-dependent azoreductase.

One or more of the enzymes used in the process of the invention may require one or more cofactors.

As used herein, the term “cofactor” refers to a non-protein chemical compound that is required for an enzyme's activity as a catalyst. Cofactors can be divided into two types, either inorganic ions, or complex organic molecules called coenzymes. For sake of clarity, in the present description, the term “cofactor” is used to indicate any non-protein chemical compound that is required for an enzyme's activity, according to the protein of the invention, without restriction to a specific chemical class of molecules, i.e., including both organic and inorganic molecules.

According to embodiments, cofactor regenerating enzymes may be used to regenerate the cofactor(s) which may be needed by the enzymes used in the process of the invention.

In this case, advantageously, expensive cofactors (e.g., NADPH) are regenerated by consuming inexpensive cofactors (such as glucose, phosphite or formate).

According to embodiments, the step of hydroxylating indole (or an indole derivative) in the presence of at least an oxidizing enzyme, to obtain indoxyl or an indoxyl derivative, may be carried out in the presence of at least an enzyme suitable for regenerating the cofactor, required by the oxidizing enzyme. For example, when the oxidizing enzyme is a monooxygenase, NADPH may be used as cofactor.

According to embodiments, the step of converting indoxyl (or indoxyl derivative), in the presence of at least a reducing enzyme, to leuco indigo or to a leuco form of an indigo derivative, may be carried out in the presence of at least an enzyme suitable for regenerating the cofactor, required by the reducing enzyme. For example, when the reducing enzyme is an azoreductase, NADH may be used as cofactor.

According to embodiments, the oxidizing enzyme and/or the reducing enzyme is coupled to a cofactor-regenerating enzyme, preferably fused to a cofactor-regenerating enzyme.

In other words, according to embodiments, the oxidizing enzyme may be a fusion enzyme wherein the oxidizing enzyme is fused to a cofactor-regenerating enzyme, and/or the reducing enzyme may be a fusion enzyme wherein the reducing enzyme is fused to a cofactor-regenerating enzyme.

According to embodiments, the cofactor-regenerating enzyme is selected from the group consisting of glucose dehydrogenase (GDH), phosphite dehydrogenase (PTDH), and formate dehydrogenase (FDH), and preferably is PTDH. In embodiments, the cofactor-regenerating enzyme is suitable to regenerate NADPH and/or NADH cofactor.

According to embodiments, when, for example, the oxidizing enzyme is mFMO and the cofactor-regenerating enzyme is PTDH, the step of hydroxylating indole or an indole derivative to obtain indoxyl or indoxyl derivative may be performed using the fusion enzyme PTDH-mFMO.

According to embodiments, when, for example, the reducing enzyme is AzoA and the cofactor-regenerating enzyme is PTDH, the step of converting indoxyl or an indoxyl derivative to leuco indigo or the leuco form of the indigo derivative may be performed using the fusion enzyme PTDH-AzoA.

For example, a PTDH-AzoA fusion enzyme may have the following sequence:

(SEQ. ID NO. 10)
MGSSHHHHHHSSGLVPRGSHMLPKLVITHRVHEEILQLLAPHCELITNQT
DSTLTREEILRRCRDAQAMMAFMPDRVDADFLQACPELRVIGCALKGFDN
FDVDACTARGVWLTFVPDLLTVPTAELAIGLAVGLGRHLRAADAFVRSGK
FRGWQPRFYGTGLDNATVGFLGMGAIGLAMADRLQGWGATLQYHARKALD
TQTEQRLGLRQVACSELFASSDFILLALPLNADTLHLVNAELLALVRPGA
LLVNPCRGSVVDEAAVLAALERGQLGGYAADVFEMEDWARADRPQQIDPA
LLAHPNTLFTPHIGSAVRAVRLEIERCAAQNILQALAGERPINAVNRLPK
ANPAADSRSAAGMTKVLYITAHPHDDTQSFSMAVGKAFIDTYKEVNPDHE
VETIDLYIEDIPHIDVDVFSGWGKLRSGQGFDQLSSDEKAKVGRLSELCE
QFVSADKYIFVSPLWNFSFPPVLKAYIDSVAVAGKTFKYTEQGPVGLLTD
KKALHIQARGGIYSEGPAAQMEMGHRYLSIIMQFFGVPSFDGLFVEGHNA
MPDKAQEIKEKAVARAKDLAHTF.

Fusion enzymes suitable to be used in the process of the invention may be produced according to techniques that are known, per se, in the art.

According to embodiments, a suitable fusion enzyme may have a sequence having sequence identity of at least 80% with respect to SEQ. ID NO. 10.

Advantageously, according to embodiments of the invention, at least two enzymes selected from the group comprising said oxidizing enzyme, said reducing enzyme, said tryptophanase and said tryptophan halogenase may be coupled together, preferably fused together.

Advantageously, when the oxidizing enzyme is coupled with a cofactor-regenerating enzyme, an enzymatic complex including a tryptophanase, an oxidizing enzyme and a cofactor regenerating enzyme may be used. For example, a fusion enzyme including a tryptophanase, an oxidizing enzyme and a cofactor regenerating enzyme fused together may be used in the process of the invention. In this case, according to embodiments of the invention, advantageously, tryptophan may be converted into leuco indigo in a particularly fast and effective way.

For example, a suitable fusion enzyme including a tryptophanase, an oxidizing enzyme and a cofactor regenerating enzyme fused together may be tryptophanase-PTDH-mFMO.

As above discussed, according to embodiments of the process of the invention, indole or indole derivatives may be obtained by converting tryptophan, or a tryptophan derivative, in the presence of a tryptophanase, and PLP may be used as cofactor in the reaction catalyzed by the tryptophanase.

According to embodiments of the process of the invention, the indole derivative is a halogenated derivative of indole, obtainable by tryptophanase-catalyzed conversion of a halogenated derivative of tryptophan. Halogenated derivatives of tryptophan may be obtained by enzymatic halogenation of tryptophan, through a halogenase-catalyzed reaction.

According to embodiments, the halogenation of tryptophan to obtain its halogenated derivatives, i.e., halogenated tryptophan, may be carried out in the presence of a flavin reductase and a NAD(P)H regenerating enzyme, said NAD(P)H regenerating enzyme being preferably selected from the group consisting of glucose dehydrogenase (GDH), phosphite dehydrogenase (PTDH), and formate dehydrogenase (FDH). Preferably, the NAD(P)H regenerating enzyme is PTDH.

Flavin reductases (EC 1.5.1.30) are known enzymes, that catalyzes the following reaction:

flavin+NADPH+H⁺→reduced flavin+NADP+H⁺

while NAD(P)H regenerating enzymes are enzymes that produce NADH or NADPH, such as GDH, PTDH and FDH. For example, tryptophan halogenase may use FAD as cofactor which may be produced by the flavin reductase that may use NADH or NADPH as cofactor. Said NADH or NADPH cofactor is, in turn, produced by the NAD(P)H regenerating enzyme, through a reaction involving inexpensive cofactors, such as glucose, phosphite and formate.

Suitable flavin reductases useful for the method of the invention can be the flavin reductases of Bacillus subtilis (BsuFRE), in particular the flavin reductases of the strain WU-S2B of Bacillus subtilis.

For example, the flavin reductase may have the following sequence:

(SEQ. ID NO. 3)
MKVLVLAFHPNMEQSVVNRAFADTLKDAPGITLRDLYQEYPDEAIDVEKE
QKLCEEHDRIVFQFPLYWYSSPPLLKKWLDHVLLYGWAYGTNGTALRGKE
FMVAVSAGAPEEAYQAGGSNHYAISELLRPFQATSNFIGTTYLPPYVFYQ
AGTAGKSELAEGATQYREHVLKSF.

According to embodiments, a suitable flavin reductase may have a sequence having sequence identity of at least 80% with respect to SEQ. ID NO. 3.

According to embodiments, the flavin reductase and the NAD(P)H regenerating enzyme may be coupled, preferably fused together, to obtain a fusion enzyme including a flavin reductase enzyme and the NAD(P)H regenerating enzyme. For example, if the flavin reductase enzyme is BsuFRE and the NAD(P)H regenerating enzyme is PTDH, the fusion enzyme PTDH-BsuFRE may be obtained and used in the process of the invention.

According to embodiments, tryptophan may be converted to a halogenated tryptophan, in presence of a halogenase (e.g., a tryptophan halogenase), a halogen source, FAD and NADH cofactors, phosphite, a flavin reductase and a NAD(P)H regenerating enzyme (optionally fused together, for example the fusion enzyme PTDH-BsuFRE). According to embodiments, the process of the invention may further comprise a step of providing oxygen at least during the step of hydroxylating indole or an indole derivative in the presence of at least an oxidizing enzyme, to obtain indoxyl or an indoxyl derivative.

Oxidizing enzymes require oxygen, i.e. O₂, within the reaction mixture in order to catalyze the hydroxylation of indole or its derivative. The O₂ required for carrying out hydroxylation of indole (or indole derivative) can be the oxygen normally dissolved within the aqueous reaction mixture. In embodiments, oxygen can be provided to the reaction mixture, e.g., into the reactor wherein the one-pot conversion from indole (or tryptophan, or derivatives thereof) into leuco indigo (or the leuco form of indigo derivatives) is carried out.

According to embodiments, by varying oxygen concentration during the process of the invention, different amounts of indoxyl (or derivatives thereof) may be obtained. Advantageously, oxygen concentration may be monitored and controlled during the process of the invention, so that oxygen may be added, when required, in order to adjust the concentration of oxygen in the reaction mixture as required.

According to embodiments, leuco indigo or the leuco form of indigo derivatives may be provided to at least part of a textile by dipping dwelling, foaming, exhausting or spraying. In embodiments, said dipping, dwelling, foaming, exhausting or spraying may be carried out in inert or substantially inert atmosphere (e.g., under nitrogen or ozone) or in presence of air, e.g., open air. Dipping dwelling, foaming, exhausting and spraying and techniques that are known, per se, in the art. For example, in embodiments, a textile, or a part of a textile, may be dipped in the reactor wherein the process of conversion of indole (or tryptophan, or their derivatives) into leuco indigo (or the leuco form of indigo derivatives) is carried out.

In other words, leuco indigo or the leuco form of indigo derivatives may be provided to at least part of a textile by dipping the textile in the reactor wherein the process of conversion of indole into leuco indigo is carried out as a one-pot process.

According to an aspect, the process of the invention includes a step of oxidizing at least part of the leuco indigo or the leuco form of said indigo derivative provided to the textile, so that indigo or an indigo derivative is produced on said textile, to dye at least part of the textile.

According to embodiments, the step of oxidizing leuco indigo (or the leuco form of an indigo derivative) to indigo (or indigo derivative) may be carried out by aerial oxidation. In other words, a textile that has been provided with leuco indigo (or the leuco form of an indigo derivative) may be exposed to air, so that oxygen in the air oxidizes such leuco indigo (or the leuco form of an indigo derivative) to indigo (or indigo derivative), thereby dyeing the textile.

In embodiments, the step of oxidizing leuco indigo (or the leuco form of an indigo derivative) may be carried out by chemical oxidation, or by drying. For example, according to embodiments, a textile that has been provided with leuco indigo (or the leuco form of an indigo derivative) may be exposed to air, exposed to chemical oxidation, and/or dried so that leuco indigo (or the leuco form of an indigo derivative) is converted to indigo (or indigo derivative), thereby dyeing the textile.

Advantageously, through the process of the invention, textiles having different intensity and/or different shades of color can be obtained. For example, according to embodiments, leuco indigo or the leuco form of indigo derivatives may be provided to a textile more than once, for example, by dipping more than once the textile in a reactor containing leuco indigo (or leuco indigo derivative(s)). For example, a textile may be impregnated with a solution of leuco indigo (or the leuco form of and indigo derivative), exposed to air so that leuco indigo is oxidized to indigo, and impregnated with leuco indigo solution and exposed to air again, to increase the amount of indigo on the textile. For example, in embodiments, a textile, or a part of a textile, may be dipped more than once in the same reactor, e.g., in the reactor wherein the conversion of indole (or tryptophan, or their derivatives) into leuco indigo (or the leuco form of indigo derivatives) is carried out as a one-pot process.

According to embodiments, the concentration of leuco indigo (or the leuco form of indigo derivatives) in the reaction mixture or solution may be adjusted before providing leuco indigo (or the leuco form of indigo derivatives) to a textile.

According to embodiments, an apparatus suitable to carry out the invention method comprises at least a reactor containing enzymes, wherein said enzymes include an oxidizing enzyme and a reducing enzyme. According to embodiments, the enzymes may also include a tryptophanase and optionally a tryptophan halogenase.

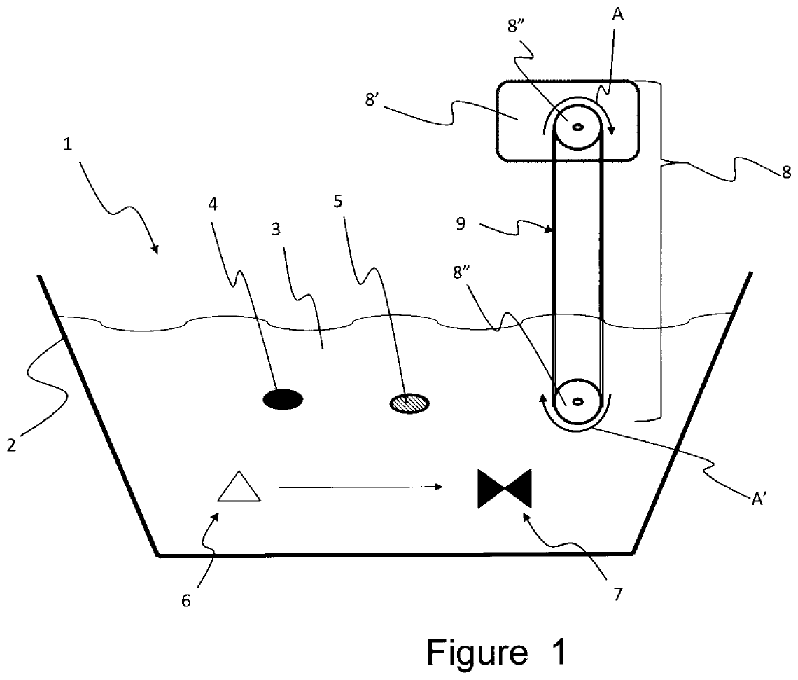

FIG. 1 schematically shows an apparatus 1 for carrying out the process of the invention, comprising a reactor 2 containing a reaction mixture 3 including enzymes, i.e., an oxidizing enzyme 4, and a reducing enzyme 5. The oxidizing enzyme 4 is preferably a monooxygenase and the reducing enzyme 5 is preferably an azoreductase. The apparatus of FIG. 1 was used to carry out the laboratory example discussed here below.

The reaction mixture 3 includes indole 6, schematically represented as a triangle in the present figures, which is converted into leuco indigo 7, schematically represented as a double triangle in the present figures, in presence of oxidizing enzymes 4 and reducing enzymes 5. In particular, indole 6 is hydroxylated in the presence of at least an oxidizing enzyme 4, to obtain indoxyl. Indoxyl is then converted to leuco indigo in the presence of at least one reducing enzyme 5. The reaction mixture 3 may further include, for example, a suitable buffer, one or more cofactors, one or more cofactor regenerating enzymes (e.g., PTDH) and optionally a catalase.

An exemplary reaction mixture 3 may include, in a suitable buffer (e.g., potassium-phosphate buffer), an oxidizing enzyme 4 (e.g., mFMO), NADPH, phosphite, phosphite dehydrogenase (PTDH), NADH, a reducing enzyme 5 (e.g., AzoA), and optionally a catalase. Indole, in presence of, for example, an oxidizing enzyme, NADPH and O₂ is converted into indoxyl, with the production of NADP⁺. Phosphite dehydrogenase (PTDH) may be used to recycle NADP⁺ to NADPH in presence of phosphite. Indoxyl, is converted to leuco indigo in the presence of a reducing enzymes. The conversion of indoxyl to leuco indigo may involve the conversion of NADH to NAD⁺.

According to embodiments, the oxidizing enzyme 4 and the reducing enzyme 5 may be provided as enzymes fused with a cofactor regenerating enzyme. Such fusion enzymes may be, for example, PTDH-mFMO and PTDH-AzoA.

According to embodiments, the reaction mixture 3 may comprise tryptophan instead of at least part of the indole, and a tryptophanase to convert the tryptophan to indole. According to embodiments, when tryptophan is used, the reaction mixture 3 may further include a tryptophan halogenase to obtain halogenated tryptophan, which is converted to the leuco form of the corresponding halogenated derivative of indigo. The obtained leuco indigo, e.g. as the leuco indigo containing reaction mixture, produced in the reactor 2 may be applied to a textile or may be removed from the reactor 2 and stored.

In embodiments, the reaction mixture containing the obtained leuco indigo may be removed from reactor 2, in the form of a reaction mixture which does not or substantially does not include enzymes, and placed into a chamber, optionally adjusting the concentration of leuco indigo, e.g., reducing the concentration of leuco indigo in the mixture.

According to embodiments, a reaction mixture including leuco indigo, which does not or substantially does not include enzymes, may be obtained by using immobilized enzymes, as above defined.

According to embodiments, enzymes may be removed from the leuco indigo containing reaction mixture by filtering techniques such as, for example, using tangential flow filtration devices (TFF). Tangential flow filtration devices (TFF) are devices that are known, per se, in the art. Such devices include a filter that allows the passage of small molecules (e.g., leuco indigo), but not enzymes.

According to embodiments, the concentration of leuco indigo (or the leuco form of indigo derivatives) in the solution may be adjusted before providing leuco indigo (or the leuco form of indigo derivatives) to a textile. Preferably, the reaction mixture in the reactor containing the enzymes has a high concentration of leuco indigo. After removal of the enzymes, remaining reaction mixture is fed to a chamber to be stocked or diluted to the required concentration e.g. for dyeing.

FIG. 1 schematically shows an apparatus 1 further comprising a device 8 for dyeing a piece of textile by repeated immersion (dipping) and removal of the textile 9 in the reaction mixture 3 provided within the reactor 2. According to the embodiment of FIG. 1, the device 8 includes a motor 8′ and two rollers 8″. A first roller 8″ is connected to the motor 8′, outside the reactor 2; and a second roller 8″ is placed inside the reactor 2.

The motor 8′ of the device 8 is configured so that at least the roller 8″ connected to the motor 8′ rotates, so that the textile 9 is immersed in and removed from the reaction mixture 3 following the direction represented by arrows A and A′ in FIG. 1. When the textile 9 is immersed in the reaction mixture 3, the textile 9 is provided, e.g., impregnated, with the solution containing leuco indigo 7. Subsequently, when the textile 9 is removed from the reaction mixture 3, it is exposed to air, so that oxidation of at least part of the leuco indigo into indigo occurs, dyeing at least part of the textile 9. The textile 9 may be, for example, a fabric a yarn or a bundle of yarns (rope). According to the embodiment schematically represented in FIG. 1, the textile 9 may be immersed into and removed from the same reaction mixture 3 more than once to increase the amount of indigo on the textile 9. For example, a textile 9 may be immersed in the reaction mixture 3 to be impregnated with the solution including leuco indigo 7, and then exposed to air so that leuco indigo 7 is oxidized to indigo on the textile 9. The textile 9 may be immersed and removed from the same reaction mixture 3 several times, so that at each immersion new leuco indigo 7 is provided to the textile 9 and then converted to indigo, to increase the amount of indigo on the textile 9.

In other examples, the textile may be impregnated with leuco indigo solution by dipping in a first reactor wherein the conversion of indole (or tryptophan, or their derivatives) into leuco indigo (or the leuco form of indigo derivatives) is carried out, and after oxidation of leuco indigo, impregnated again, e.g., with a solution including leuco indigo (or a leuco form of an indigo derivative), by dipping in a second or further reactor(s) wherein the conversion of indole (or tryptophan, or their derivatives) into leuco indigo (or the leuco form of indigo derivatives) is carried out.

According to embodiments, in the process of the invention the textile is dipped in succession in a plurality of reactors containing reaction mixtures including leuco indigo (or leuco form of said indigo derivative), wherein said textile is exposed to air between each two dipping steps.

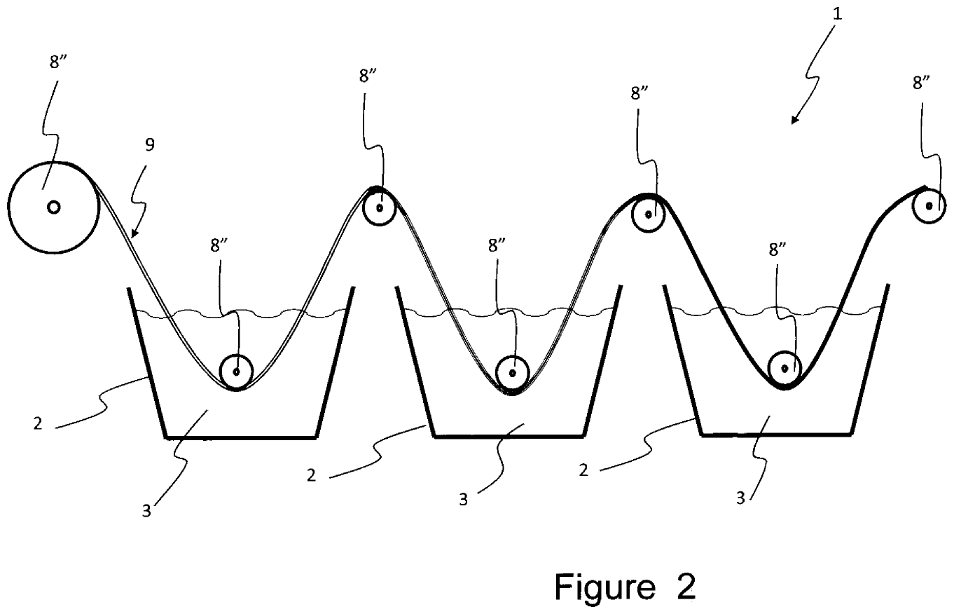

FIG. 2 schematically shows an apparatus 1 for carrying out the process of the invention, comprising a plurality of reactors 2, each one containing a reaction mixture 3. In particular, FIG. 2 shows three reactors 2, each one containing a reaction mixture 3. Each reaction mixture 3 contains enzymes, including an oxidizing enzyme 4, and a reducing enzyme 5 (not shown in FIG. 2).

The description of the exemplary reaction mixture 3 made with reference to FIG. 1, also applies to the reaction mixtures 3 schematically represented in FIG. 2. In embodiments, the different reactors may contain the same reaction mixture 3 or different reaction mixtures 3.

In the embodiment of FIG. 2 the textile (e.g. a rope of yarns) is moved from a reactor to the next one in a way known per se in the art of indigo dyeing, e.g. by using a plurality of rollers 8″ placed both outside and inside the reactors 2 in a configuration similar to those used for a indigo dyeing process according to the prior art. In other words, in the invention apparatus the plurality of reactors 2 replaces previously known dyeing baths.

According to the embodiment of FIG. 2, the textile 9, is guided by the rollers 8″ to be immersed and removed from the first reaction mixture 3, then immersed in the second reaction mixture 3 and then immersed and removed from the third the reaction mixture 3. When the textile 9 is immersed in the first reaction mixture 3, the textile 9 is provided, e.g., impregnated, with a first amount of leuco indigo 7 solution. Subsequently, when the textile 9 is removed from the first reaction mixture 3, it is exposed to air, so that oxidation of at least part of the first amount of leuco indigo occurs, so the textile is provided with a first amount of indigo, whereby dyeing at least part of the textile 9. Subsequently, the textile 9 is immersed in the second reaction mixture 3, so that the textile 9 is impregnated, with a second amount of leuco indigo 7 solution, and removed so that the textile 9 is provided with a second amount of indigo. According to the embodiment of FIG. 2, a third cycle of immersion and removal from a reaction mixture 3 is carried out, so that the textile 9 is provided with a third amount of leuco indigo and, therefore, a third amount of indigo.

FIG. 2 schematically shows the change in color of the textile 9, which occurs when the textile is exposed to air after having being immersed in the reaction mixtures 3 in the different reactors 2.

According to embodiments, if the reaction mixtures 3 include different reactants (e.g., indole and at least one indole derivative) different leuco forms of indigo derivatives may be produced in one or more reactors 2, the textile 9 may be provided with at least one indigo derivative in addition to or as an alternative to indigo.

According to embodiments of the process of the invention, advantageously, the amount of one dye, e.g., indigo, on the textile may be increased. Also advantageously, when different leuco forms are used, the textile may be provided with more than one dye to obtain a required final colour for the textile.

According to an aspect of the present invention, the soluble leuco indigo or leuco form of an indigo derivative, is obtained by means of a cascade of enzymatic reactions steps, starting from indole or tryptophan, or their derivatives, to obtain leuco indigo (or the leuco form of one or more indigo derivatives). Leuco indigo (or the leuco form of one or more indigo derivatives) is oxidized to produce indigo (or one or more indigo derivatives) through, for example, a spontaneous oxidation reaction occurring when a textile impregnated with a solution including said leuco indigo is, for example, exposed to air. After that the textile is provided with indigo or a derivative thereof, it may be optionally washed and/or rinsed and dried.

According to embodiments, the textile, i.e., the textile article, is selected from a yarn, a fabric or a garment.

According to embodiments, a textile article, preferably selected from the group consisting of a yarn, a fabric and a garment, may be provided with leuco indigo and/or leuco forms of one or more indigo derivatives, whereby at least part of the leuco indigo and/or leuco forms of one or more indigo derivatives is oxidized (for example, by exposure to air) to produce indigo and/or one or more indigo derivatives onto the textile.

Another object of the present invention is a dyed textile as obtainable according to the process of the invention.

According to embodiments, the dyed textile is an indigo dyed textile, e.g., an indigo dyed yarn, an indigo dyed fabric or an indigo dyed garment. According to embodiments, the dyed textile is a Tyrian purple dyed textile, e.g., a Tyrian purple dyed yarn, a Tyrian purple dyed fabric or a Tyrian purple dyed garment.

According to embodiments, when the textile is a yarn, dyed yarns may be used in to production of articles, such as fabrics and clothing articles, e.g., garments. According to embodiments, when the textile is a fabric, the dyed fabric may be tailored into a garment, or may be included into a garment.

A further object of the present invention is a method for the production of leuco indigo or the leuco form of an indigo derivative by enzymatic synthesis that comprises the following steps:
a′) providing indole or an indole derivative, optionally by converting tryptophan or a tryptophan derivative in the presence of at least a tryptophanase to said indole or indole derivative;
b′) hydroxylating the indole or the indole derivative obtained in step a′) in the presence of at least an oxidizing enzyme, to obtain indoxyl or an indoxyl derivative; and
c′) converting the indoxyl or the indoxyl derivative obtained in step b′) in the presence of at least a reducing enzyme, to leuco indigo or the leuco form of an indigo derivative.

Advantageously, according to embodiments, the method of the invention allows the synthesis of leuco indigo or the leuco form of an indigo derivative, preferably starting from tryptophan or a tryptophan derivative, by means of a cascade of enzymatic reaction steps.

According to embodiments, the method of the invention further comprises a step of oxidizing the leuco indigo or the leuco form of the indigo derivative to obtain indigo or said indigo derivative.

The method of the invention is particularly advantageous to produce leuco indigo and leuco forms of indigo derivatives, as well as indigo and/or indigo derivatives, such as Tyrian purple, in a cost effective way.

Also, advantageously, the method of the invention allows the manufacturing of leuco indigo and leuco forms of indigo derivatives, as well as indigo and/or indigo derivatives on an industrial scale.

In the present description, information provided with reference to the process for dyeing textiles, including enzymes used as well as reagents and obtained products, also apply to the method for the production of leuco indigo or the leuco form of an indigo derivative, as well as indigo and indigo derivatives, by enzymatic synthesis, which is also an object of the present invention.

According to the present invention, all the enzymes used in the process for dyeing textiles, as well as in the method for the production of leuco indigo or the leuco form of an indigo derivative by enzymatic synthesis, may be genetically engineered in order to provide the enzymes with, for example, additional functional features and/or improved activity.

Advantageously, according to embodiments, oxidation of leuco indigo or the leuco form of the indigo derivative, to indigo or the indigo derivative may be performed after that leuco indigo has been provided to a support, e.g., a textile article, such as a fabric. According to embodiments, the step of oxidizing leuco indigo (or the leuco form of an indigo derivative) to indigo (or indigo derivative) may be carried out by aerial oxidation. For example, a textile that has been provided with leuco indigo may be exposed to air, so that oxygen in the air oxidizes such leuco indigo to indigo on the surface of the textile.

Advantageously, according to embodiments, tryptophan, can be used as a starting compound to enzymatically produce leuco indigo and leuco forms of indigo derivatives, as well as indigo and indigo derivatives. Advantageously, the use of tryptophan as starting compound allows for a cost effective production of indigo and/or indigo derivatives, and leuco form thereof.

According to embodiments, the tryptophan derivative of step a′) of the process of the invention is a halogenated derivative of tryptophan. Preferably, the halogenated tryptophan is 6-bromotryptophane.

According to embodiments, when the tryptophan derivative is a halogenated derivative, the method of the invention further comprises a step of: i) halogenating tryptophan, to obtain the halogenated derivative of tryptophan, in the presence of at least a tryptophan halogenase and a halogen source. Preferably, the halogen source is halogen bromine.

According to embodiments, the enzymes employed in the method of the invention, as well as in the process of the invention, may be isolated enzymes, preferably purified or semi-purified enzymes. Enzymes may be isolated and/or purified from host cells, e.g., from bacterial cells, and host organisms according to techniques that are known in the art.

According to embodiments, the tryptophanase and/or the oxidizing enzyme and/or the reducing enzyme and/or the tryptophan halogenase are isolated enzymes.

According to embodiments, the tryptophanase and/or the oxidizing enzyme and/or the reducing enzyme and/or the tryptophan halogenase are immobilized enzymes. According to embodiments, steps b′), c′) and, optionally, said step a′) and said step of halogenating tryptophan, are carried out in a single reactor, i.e., as a one-pot process. According to embodiments, when the halogen source is halogen bromine, the halogenated derivative of tryptophan is preferably 6-bromotryptophan and the indigo derivative is preferably Tyrian purple.

According to embodiments, the method of the invention may be carried out in the presence of a textile, whereby at least part of said textile is provided at least in part with leuco indigo and/or with said leuco form of said indigo derivative.

In this case, advantageously, the step of oxidizing leuco indigo (or the leuco form of an indigo derivative) to indigo (or indigo derivative) may be carried out by aerial oxidation, e.g., by exposing the textile that has been provided with leuco indigo to air, so that oxygen in the air oxidizes such leuco indigo to indigo on the surface of the textile.

According to embodiments, the step of oxidizing the leuco indigo or the leuco form of an indigo derivative to obtain indigo or said indigo derivative may be performed in the presence of a textile, so that at least part of the indigo or indigo derivatives that is obtained is deposited onto the textile. In other words, for example, leuco indigo may be provided to a textile and subsequently oxidized to obtain indigo, so that at least part of the textile is dyed.

According to embodiments, the method of the invention can be carried out in one reactor, whereby providing a one pot reaction.

According to an aspect, the present invention relates to an apparatus for carrying out the method of the invention, comprising a reactor containing enzymes, wherein said enzymes include a reducing enzyme, preferably a monooxygenase, and a reducing enzyme, preferably an azoreductase, preferably also a tryptophanase, and optionally also a tryptophan halogenase.

Advantageously, the apparatus of the invention allows for the production of leuco indigo or leuco froms of indigo derivatives, starting from indole (or indole derivatives) or, preferably, from tryptophan or a tryptophan derivative.

Indigo or indigo derivatives may be obtained from leuco indigo (or leuco form of indigo derivatives) according to standard techniques, such as, for example, standard exposure to air. Advantageously, when a textile is provided with leuco indigo and exposed to air, oxygen in the air oxidizes such leuco indigo to indigo on the surface of the textile.

The method of the invention, as well as the process for dyeing textiles according to the invention, may be carried out in an aqueous medium. Such aqueous medium may have a neutral or slightly basic pH, such as 7.0 to 10, preferably 7.4 to 9. Such aqueous medium can thus comprise a buffering agent, for example a potassium phosphate buffer or a Tris HCl buffer. Some tryptophan derivatives, such as 6-bromotryptophan, are poorly soluble in aqueous medium, and the method of the invention can be carried out with such tryptophan derivatives suspended in the aqueous medium.

Step a′) involves the cleavage of a carbon-carbon bond on tryptophan or on the derivative thereof in the presence of a tryptophanase.

As above mentioned, tryptophanases are known enzymes that cleave a carbon-carbon bond of tryptophan, releasing indole. They may use pyridoxal phosphate (PLP) as cofactor. A tryptophanase suitable to be used in the method of the invention the tryptophanase of Escherichia coli NEB® 10β.

PLP can be optionally added to the reaction mixture of step a′) to improve the yield of the conversion of tryptophan or of its derivatives.

Step b′) of the method of the invention involves the hydroxylation at least on the carbon in position 3 of the indole or its derivative obtained from step a′) in the presence of oxidizing enzyme and O₂. Step b′) thus provides indoxyl or indoxyl derivatives. Suitable oxidizing enzymes are the ones as described above, e.g. microbial FMO (mFMO), such as microbial FMO from Methylophaga sp strain SK1 and Baeyer-Villiger monooxygenase.

Oxidizing enzymes require O₂, i.e. oxygen, within the reaction mixture in order to catalyze the hydroxylation of indole or its derivative. The O₂ required for carrying out step b′) of the method of the invention can be the oxygen normally dissolved within the aqueous reaction mixture; if necessary, concentration of O₂ in the reaction mixture can be adjusted in order to, for example, increase conversion of indole or its derivatives into indoxyl or its derivatives.

Oxygen, i.e. O₂, is also required to convert leuco indigo (or leuco-indigo derivatives) to indigo (or indigo derivatives). For example, indigo may be obtained from leuco indigo through a non-enzymatic reaction, e.g., by exposure to air.

According to embodiments, the tryptophan derivative of step a′) is a halogenated tryptophan that is obtained through a step of i) halogenating tryptophan in the presence of at least a tryptophan halogenase.

As above discussed, tryptophan halogenases are known enzymes able to catalyze the halogenation of tryptophan in various positions. Tryptophan halogenases are usually flavin-dependent halogenases, i.e. they use FAD or FADH₂ as a cofactor. Suitable tryptophan halogenases according to the method of the invention are tryptophan halogenases, such as the tryptophan halogenase of Streptomyces violaceusniger. According to embodiments, the tryptophan halogenase is the tryptophan halogenase of the strain SPC6 of Streptomyces violaceusniger.

For example, the tryptophan halogenase may have sequence SEQ. ID NO. 1, above reported.

This type of tryptophan halogenase catalyze preferably the halogenation on the carbon in position 6 of tryptophan, whereby it is suitable to produce Tyrian purple (6,6′-dibromoindigo) according to the method of the invention.

Another tryptophan halogenase suitable for the method of the invention is tryptophan halogenase PrnA, preferably is the PrnA of Pseudomonas fluorescens, which catalyzes preferably the halogenation of tryptophan on the carbon in position 5 or 7 of the tryptophan.

For example, the tryptophan halogenase (PrnA) may have sequence SEQ. ID. NO. 2, above reported.

According to embodiments, the tryptophan halogenase may be a genetically modified enzyme; in other words, the tryptophan halogenase may be in a mutant form. For example, the tryptophan halogenase may be a mutant form of the tryptophan halogenase of the strain SPC6 of Streptomyces violaceusniger, or a mutant form of the tryptophan halogenase PrnA.

Step i) of halogenating tryptophan, to obtain a halogenated derivative of tryptophan, requires a halogen source within the reaction mixture in order to be carried out, as tryptophan has to react with a halogen in presence of tryptophan halogenase to be converted to a halogenated derivative of tryptophan, i.e., to halogenated tryptophan. Suitable halogen sources according to the method of the invention can be halogen salts, i.e. salts wherein the anion is halide ion. Suitable halogen salts can be magnesium, silver, sodium, potassium, lithium, and calcium halogen salts, for example NaCl, KCl, KI, LiCl, CuCl₂, CuBr₂, AgCl, CaCl₂, CaBr₂, ClF, MgCl₂, MgBr₂, etc.

Step i) of halogenating tryptophan may be carried out, according to embodiments, at temperature comprised in the range from 20° C. to 60° C., preferably from 25° C. to 40, more preferably at about 30° C., for a time comprised in the range of from 30 minutes to 4 hours, preferably from 1 hour to 3 hours, more preferably for about 2 hours. According to embodiments, cofactor regenerating enzymes may be used to regenerate the cofactor(s) which may be needed by the enzymes used in the method of the invention.

According to embodiments, step b′) may be carried out in the presence of at least an enzyme suitable for regenerating NADPH cofactor. Preferably, the enzyme suitable for regenerating NADPH cofactor is selected from the group consisting of glucose dehydrogenase (GDH), phosphite dehydrogenase (PTDH), and formate dehydrogenase (FDH) as described below, more preferably is PTDH as described below, whereby a NADPH regenerating enzyme system is provided. Advantageously, this embodiment provides for an enzyme system wherein expensive cofactors (i.e. NADPH) are regenerated by consuming cheaper cofactors (such as glucose, phosphite or formate). For example, oxidizing enzymes such as FMOs may use NADPH as cofactor which may be produced by the NADPH regenerating enzyme that uses cheap cofactors such as glucose, phosphite and formate.

In another embodiment, the halogenation of tryptophan to obtain its halogenated derivative is carried out in the presence of a flavin reductase and a NAD(P)H regenerating enzyme, preferably selected from the group consisting of glucose dehydrogenase (GDH), phosphite dehydrogenase (PTDH), and formate dehydrogenase (FDH), more preferably is PTDH, whereby a tryptophan halogenase-flavin reductase-NAD(P)H regenerating enzyme system is provided.

Flavin reductases (EC 1.5.1.30) are enzyme that catalyzes the following reaction:

flavin+NADPH+H⁺→reduced flavin+NADP+H⁺

while NAD(P)H regenerating enzymes are enzymes that produce NADH or NADPH, such as GDH, PTDH and FDH. Advantageously, this embodiment provides for an enzyme system wherein expensive cofactors (i.e. FAD and NADH or NADPH) are regenerated by consuming cheaper cofactors (such as glucose, phosphite or formate), improving the industrial feasibility of the method of the invention. For example, tryptophan halogenase may use FAD as cofactor which may be produced by the flavin reductase that may use NADH or NADPH as cofactor, which is produced by the NAD(P)H regenerating enzyme that uses cheap cofactors such as glucose, phosphite and formate.

Suitable flavin reductases useful for the method of the invention can be the flavin reductases of Bacillus subtilis, in particular the flavin reductases of the strain WU-S2B of Bacillus subtilis. For example, the flavin reductases may have the sequence SEQ. ID NO. 3, above reported.

Wild type forms of the enzymes suitable to be used in the process and the method of the invention are known, per se, in the art. For example, a suitable mMFO is the wild type form of mMFO of Methylophaga aminisulfidivorans, having the following sequence:

(SEQ. ID NO. 5)
MATRIAILGAGPSGMAQLRAFQSAQEKGAEIPELVCFEKQADWGGQWNYT
WRTGLDENGEPVHSSMYRYLWSNGPKECLEFADYTFDEHFGKPIASYPPR
EVLWDYIKGRVEKAGVRKYIRFNTAVRHVEFNEDSQTFTVTVQDHTTDTI
YSEEFDYVVCCTGHFSTPYVPEFEGFEKFGGRILHAHDFRDALEFKDKTV
LLVGSSYSAEDIGSQCYKYGAKKLISCYRTAPMGYKWPENWDERPNLVRV
DTENAYFADGSSEKVDAIILCTGYIHHFPFLNDDLRLVTNNRLWPLNLYK
GVVWEDNPKFFYIGMQDQWYSFNMFDAQAWYARDVIMGRLPLPSKEEMKA
DSMAWREKELTLVTAEEMYTYQGDYIQNLIDMTDYPSFDIPATNKTFLEW
KHHKKENIMTFRDHSYRSLMTGTMAPKHHTPWIDALDDSLEAYLSDKSEI
PVAKEA.

According to embodiments, a suitable mMFO may have a sequence having sequence identity of at least 80% with respect to SEQ. ID NO. 5.

Mutant forms of any enzyme employed in the method and the process of the invention can be used to improve yields and industrial feasibility of the method and the process of the invention. Techniques suitable to be used to produce mutant forms of enzymes are known in the art.

For example, one or more mutation may be introduced into a wild type sequence, to obtain a mutant form of the enzyme which is more thermostable, i.e., a mutant form of the enzyme having an apparent melting temperature greater than the apparent melting temperature of the wild type form of the same enzyme.

For example, it has been observed that inserting two mutations at the N-terminus M15L and S23A, in the sequence of wild type mMFO of Methylophaga aminisulfidivorans (SEQ. ID NO. 5, above reported) resulted in a 3° C. increase in apparent melting temperature.

Therefore, a suitable mMFO is the M15L/S23A mutant form of mMFO of Methylophaga aminisulfidivorans, having the following sequence:

(SEQ. ID NO. 6)
MATRIAILGAGPSGLAQLRAFQAAQEKGAEIPELVCFEKQADWGGQWNYT
WRTGLDENGEPVHSSMYRYLWSNGPKECLEFADYTFDEHFGKPIASYPPR
EVLWDYIKGRVEKAGVRKYIRFNTAVRHVEFNEDSQTFTVTVQDHTTDTI
YSEEFDYVVCCTGHFSTPYVPEFEGFEKFGGRILHAHDFRDALEFKDKTV
LLVGSSYSAEDIGSQCYKYGAKKLISCYRTAPMGYKWPENWDERPNLVRV
DTENAYFADGSSEKVDAIILCTGYIHHFPFLNDDLRLVTNNRLWPLNLYK
GVVWEDNPKFFYIGMQDQWYSFNMFDAQAWYARDVIMGRLPLPSKEEMKA
DSMAWREKELTLVTAEEMYTYQGDYIQNLIDMTDYPSFDIPATNKTFLEW
KHHKKENIMTFRDHSYRSLMTGTMAPKHHTPWIDALDDSLEAYLSDKSEI
PVAKEA.

Additionally or alternatively, mutations may be provided that improve catalytic activity of enzymes.

For example, FMO mutations selected from the group consisting of C78I, C78V, Y207W, Y207W/W319A, C78I/Y207W/W319A were found out to improve the catalyzing activity of FMO on indole.

In particular, it was observed that mutant C78I has a catalytic activity greater than the wild type form (i.e., C78I has a higher value of k_{cat} with respect to the wild type form). It was observed that the C78I mutation has an unexpected large effect on catalytic speed. In fact it was found that the C78 position is located in the second shell of the structure of the FMO enzyme.

Also, it was observed that mutant Y207W has an affinity for the substrate (i.e., indole) greater than the wild type form (i.e., Y207W has a lower value of K_{M} with respect to the wild type form).

For example, a suitable mMFO is the M15L/S23A/C78I mutant form of mMFO of Methylophaga aminisulfidivorans, having the following sequence:

(SEQ. ID NO. 7)
MATRIAILGAGSGLAQLRAFQAAQEKGAEIPELVCFEKQADWGGQWNYTW
RTGLDENGEPVHSSMYRYLWSNGPKEILEFADYTFDEHFGKPIASYPPRE
VLWDYIKGRVEKAGVRKYIRFNTAVRHVEFNEDSQTFTVTVQDHTTDTIY
SEEFDYVVCCTGHFSTPYVPEFEGFEKFGGRILHAHDFRDALEFKDKTVL
LVGSSYSAEDIGSQCYKYGAKKLISCYRTAPMGYKWPENWDERPNLVRVD
TENAYFADGSSEKVDAIILCTGYIHHFPFLNDDLRLVTNNRLWPLNLYKG
VVWEDNPKFFYIGMQDQWYSFNMFDAQAWYARDVIMGRLPLPSKEEMKAD
SMAWREKELTLVTAEEMYTYQGDYIQNLIDMTDYPSFDIPATNKTFLEWK
HHKKENIMTFRDHSYRSLMTGTMAPKHHTPWIDALDDSLEAYLSDKSEIP
VAKEA.

For example, a suitable mMFO is the M15L/S23A/Y207W mutant form of mMFO of Methylophaga aminisulfidivorans, having the following sequence:

(SEQ. ID NO. 8)
MATRIAILGAGPSGLAQLRAFQAAQEKGAEIPELVCFEKQADWGGQWNYT
WRTGLDENGEPVHSSMYRYLWSNGPKECLEFADYTFDEHFGKPIASYPPR
EVLWDYIKGRVEKAGVRKYIRFNTAVRHVEFNEDSQTFTVTVQDHTTDTI
YSEEFDYVVCCTGHFSTPYVPEFEGFEKFGGRILHAHDFRDALEFKDKTV
LLVGSSWSAEDIGSQCYKYGAKKLISCYRTAPMGYKWPENWDERPNLVRV
DTENAYFADGSSEKVDAIILCTGYIHHFPFLNDDLRLVTNNRLWPLNLYK
GVVWEDNPKFFYIGMQDQWYSFNMFDAQAWYARDVIMGRLPLPSKEEMKA
DSMAWREKELTLVTAEEMYTYQGDYIQNLIDMTDYPSFDIPATNKTFLEW
KHHKKENIMTFRDHSYRSLMTGTMAPKHHTPWIDALDDSLEAYLSDKSEI
PVAKEA.

Other mutant forms mMFO of Methylophaga aminisulfidivorans were tested, i.e.: C78L, C78A, W319A, W319F, Y207N/W319A, Y207N/W319F, Y207N/W319N, Y207N, Y207W/W319C, Y207W/W319F, Y207W/W319N, W319N, C78F/Y207N/W319A, C78F/Y207N/W319F, C78F/Y207N/W319N, C78F/Y207N, C78F/Y207W/W319F, C78F/Y207W/W319N, C78F/Y207W, C78F/W319F, C78F/W319N, C78I/Y207N/W319A, C78I/Y207N/W319F, C78I/Y207N/W319N, C78I/Y207N, C78I/Y207W/W319F, C78I/Y207W/W319N, C78I/Y207W, C78I/W319A, C78I/W319F, C78I/W319N, C78V/Y207N/W319A, C78V/Y207N/W319F, C78V/Y207N/W319N, C78V/Y207N, C78V/Y207W/W319A, C78V/Y207W/W319F, C78V/Y207W/W319N, C78V/Y207W, C78V/W319A, C78V/W319F, C78V/W319N.

For example, FMO mutations selected from the group consisting of W319A, C78I, C78I/Y207W, and C78I/Y207W/W319F were found out to improve the catalyzing activity of FMO on 6-bromoindole. Moreover, the NADPH regenerating enzyme can be a mutant that has improved NADPH production, e.g. PTDH disclosed in WO 2004/108912 A2.

The same or substantially the same mutations above discussed with reference to mFMO may be introduced in the sequence of FMO of Nitrincola lacisaponensis (NiFMO). In fact, it has been observed that NiFMO has almost identical active site as mFMO.

According to embodiments, suitable mMFOs may have a sequence having sequence identity of at least 80% with respect to SEQ. ID NO. 6, or SEQ. ID NO 7, or SEQ. ID NO. 8.

Additionally, mutant form of the AzoA reductase of Bacillus wakoensis (SEQ. ID NO. 4), above reported were tested: W60A, W60T, W60D, W60R, W60F. According to embodiments, enzymes having sequence identity of at least 80% with respect to the wild type form of any enzyme employed in the process and the method of the invention can be used, provided that such enzymes catalyze the same reaction of the wild type form.

According to embodiments, when an enzyme requires a cofactor, such enzyme may be provided as a fusion enzyme with the cofactor-regenerating enzyme.

For example, tryptophan halogenase and flavin reductase can be provided as a fusion enzyme, and FMO and NADPH regenerating enzyme can be provided as a fusion enzyme, preferably as PTDH-FMO. According to this embodiment, only three individual enzymes can be employed in the method of the invention (when optional step i) is carried out), namely a tryptophan halogenase-flavin reductase fusion enzyme, a tryptophanase and a FMO-NADPH regenerating fusion enzyme. The NADPH regenerating portion of the latter fusion enzyme is able to regenerate the NADPH required for both the FMO region and the flavin reductase region of the fusion enzyme starting from its cheap substrate, i.e. phosphite.

For example, a tryptophan halogenase-flavin reductase fusion enzyme (Thal-FRE) may have the following sequence:

(SEQ. ID NO. 9)
MGSSHHHHHHSSGLVPRGSHLNNVVIVGGGTAGWMTASYLKAAFGDRIDI
TLVESGHIGAVGVGEATFSDIRHFFEFLGLKEKDWMPACNATYKLAVRFE
NWREKGHYFYHPFEQMRSVNGFPLTDWWLKQGPTDRFDKDCFVMASVIDA
GLSPRHQDGTLIDQPFDEGADEMQGLTMSEHQGKTQFPYAYQFEAALLAK
YLTKYSVERGVKHIVDDVREVSLDDRGWITGVRTGEHGDLTGDLFIDCTG
FRGLLLNQALEEPFISYQDTLPNDSAVALQVPMDMERRGILPCTTATAQD
AGWIWTIPLTGRVGTGYVYAKDYLSPEEAERTLREFVGPAAADVEANHIR
MRIGRSRNSWVKNCVAIGLSSGFVEPLESTGIFFIHHAIEQLVKNFPAAD
WNSMHRDLYNSAVSHVMDGVREFLVLHYVAAKRNDTQYWRDTKTRKIPDS
LAERIEKWKVQLPDSETVYPYYHGLPPYSYMCILLGMGGIELKPSPALAL
ADGGAAQREFEQIRNKTQRLTEVLPKAYDYFTQSGSAAGMKVLVLAFHPN
MEQSVVNRAFADTLKDAPGITLRDLYQEYPDEAIDVEKEQKLCEEHDRIV
FQFPLYWYSSPPLLKKWLDHVLLYGWAYGTNGTALRGKEFMVAVSAGAPE
EAYQAGGSNHYAISELLRPFQATSNFIGTTYLPPYVFYQAGTAGKSELAE
GATQYREHVLKSF.

According to embodiments, a suitable fusion enzyme may have a sequence having sequence identity of at least 80% with respect to SEQ. ID NO. 9.

Advantageously, according to embodiments of the invention, at least two enzymes selected from the group comprising the oxidizing enzyme, the reducing enzyme, the tryptophanase and the tryptophan halogenase may be coupled together, preferably fused together.

Advantageously, when the oxidizing enzyme is coupled with a cofactor-regenerating enzyme, an enzymatic complex including a tryptophanase, an oxidizing enzyme and a cofactor regenerating enzyme may be used. For example, a fusion enzyme including a tryptophanase, an oxidizing enzyme and a cofactor regenerating enzyme fused together may be used in the method of the invention. In this case, according to embodiments of the invention, advantageously, tryptophan may be converted into leuco indigo in a particularly fast and effective way.

For example, a suitable fusion enzyme including a tryptophanase, an oxidizing enzyme and a cofactor regenerating enzyme fused together may be tryptophanase-PTDH-mFMO.

### EXPERIMENTAL SECTION

### Example 1

Materials and Methods

Indigo dyeing of a cotton belt according to the process of the invention was carried out using the fusion enzyme PTDH-mFMO (oxidizing enzyme mFMO fused to cofactor regenerating enzyme PTDH) and PTDH-AzoA (reducing enzyme AzoA fused to cofactor regenerating enzyme PTDH).

Cotton belt was stitched manually from several pieces of cotton to give final dimensions of 2×20 cm.

The apparatus for this experiment is configured as schematically illustrated in FIG. 1. In particular, the device 8 for cyclic or alternate immersion (dipping) and removal of the textile in the reaction mixture included a peristaltic pump as motor 8′, having a head adapted to be configured as a roller 8″ to provide rotational motion for cotton belt, which was immersed in the reaction mixture (2 cm immersion).

Cotton belt is rotated by the heat of the peristaltic pump so that the cotton rotates inside and outside the reaction mixture. The cotton belt is thus impregnated with a solution comprising leuco indigo when it is immersed in the reaction mixture, and subsequently exposed to air when it is not immersed in the reaction mixture. Cyclic immersion of the cotton belt in the reaction mixture and exposition to air was continued for 165 minutes.

The reaction mixture (100 mL) contained, in a single reactor:
PTDH-mFMO (1.5 μM),
PTDH-AzoA (0.6 μM),
NADH (0.2 mM),
NADPH (0.2 mM),
Na-phosphite (20 mM),
indole (5 mM) and
catalase
in 120 mL potassium-phosphate buffer 50 mM pH 8.5.

Results

Reaction mixture started turning yellow already after 20-30 min, which is indicative for the presence of leuco-indigo. Blue color in the mixture, due to the presence of indigo, was not observed at any moment of the reaction. Without being bound to a specific scientific explanation, is has been hypothesized that the indigo which is produced by the hydroxylation of indole by mFMO and subsequent dimerization, is immediately and continuously reduced into leuco indigo by AzoA. Appearance of the blue color on cotton belt was evident after 45 min since the reaction started and gained darker shades in the following 2 h. Reaction was stopped. It was noticed that when the reaction mixture was stored at 4° C. for 7 days in this period the mixture remained substantially yellow. This experiment shows that the enzymatic textile dyeing is possible and also that the leuco indigo solution of the reaction mixture is stable and can be stored. Alternatively, the leuco-indigo could be produced using tryptophanase, monooxygenase and azoreductase (optionally immobilized). In this case, tryptophan can be used as starting material of the process. Optional immobilization of the enzymes allows to maximize the re-use of enzymes.

For example, recombinant tryptophanase from E. coli can be used efficiently for this process. This enzyme can be produced easily according to techniques that are, per se, known, and requires addition of pyridoxal-5-phosphate (PLP) in the reaction mixture. Moreover, the tryptophanase from E. coli also accepts halogenated tryptophan and therefore same process can be used for synthesis and dyeing of fabric with halogenated indigo derivatives.

1. A process for dyeing textiles comprising the following steps:
a) hydroxylating indole or an indole derivative in the presence of at least an oxidizing enzyme, to obtain indoxyl or an indoxyl derivative;
b) converting said indoxyl or said indoxyl derivative to leuco indigo or to a leuco form of an indigo derivative in the presence of at least a reducing enzyme;
c) providing at least said leuco indigo or said leuco form of said indigo derivative to at least part of a textile; and
d) oxidizing at least part of said leuco indigo or said leuco form of said indigo derivative, so that indigo or an indigo derivative is produced on said textile, to dye at least part of said textile.

2. The process according to claim 1, further comprising a step of converting tryptophan or a tryptophan derivative in the presence of at least a tryptophanase, to obtain said indole or said indole derivative.

3. The process according to claim 2, wherein said tryptophan derivative is a halogenated derivative of tryptophan, and wherein said process further comprises a step of halogenating tryptophan in the presence of at least a tryptophan halogenase and a halogen source, to obtain said halogenated derivative of tryptophan.

4. The process according to claim 1, wherein said steps a) and b) are carried out as a one-pot process.

5. The process according to claim 3, wherein said oxidizing enzyme, said reducing enzyme, said tryptophanase and said tryptophan halogenase are isolated enzymes.

6. The process according to claim 3, wherein said tryptophan derivative is 6-bromotryptophan and said indigo derivative is Tyrian purple.

7. The process according to claim 1, wherein said oxidizing enzyme is preferably a monooxygenase, flavin-containing monooxygenase (FMO).

8. The process according to claim 1, wherein said reducing enzyme is an azoreductase.

9. The process according to claim 1, wherein said oxidizing enzyme and said reducing enzyme are coupled to a cofactor-regenerating enzyme, wherein said cofactor-regenerating enzyme is selected from glucose dehydrogenase (GDH), phosphite dehydrogenase (PTDH), formate dehydrogenase (FDH), and mixtures thereof.

10. The process according to claim 1, wherein said oxidizing enzyme is the fusion enzyme PTDH-mFMO and said reducing enzyme is PTDH-AzoA.

11. The process according to claim 1, wherein said leuco indigo or leuco form of an indigo derivative is provided to at least part of said textile by a method selected from dipping, dwelling, foaming, exhausting or spraying or a combination thereof, wherein said textile is exposed to air, or exposed to chemical oxidation, or dried after said textile has been provided with said leuco indigo or said leuco form of an indigo derivative.

12. (canceled)

13. The process according to claim 11, wherein said leuco indigo or said leuco form of an indigo derivative is provided to said textile by dipping and/or dwelling said textile in succession in a plurality of reactors or chambers containing reaction mixtures or aqueous solutions including leuco indigo or said leuco form of indigo derivative, and wherein said textile is exposed to air between each two dipping steps.

14. The process according to claim 1, wherein at least one of said oxidizing enzyme, reducing enzyme, tryptophanase, tryptophan halogenase is an immobilized enzyme.

15. The process according to claim 1, wherein said textile is selected from a yarn, a fabric or a garment.

16. (canceled)

17. A method for the production of leuco indigo or the leuco form of an indigo derivative by enzymatic synthesis that comprises the following steps:
a′) providing indole or an indole derivative;
b′) hydroxylating said indole or said indole derivative obtained in step a′) in the presence of at least an oxidizing enzyme, to obtain indoxyl or an indoxyl derivative; and
c′) converting said indoxyl or said indoxyl derivative obtained in step b′) in the presence of at least a reducing enzyme, to leuco indigo or to the leuco form of an indigo derivative.

18. The method according to claim 17, further comprising the step of converting tryptophan or a tryptophan derivative to said indole or said indole derivative in the presence of at least a tryptophanase.

19. The method according to claim 18, wherein at least one of said tryptophanase, oxidizing enzyme, reducing enzyme, and tryptophan halogenase is an isolated enzyme.

20. The method according to claim 17, wherein said step b′) and c′) are carried out in the same reactor as a one-pot process.

21. An apparatus for carrying out a process according to claim 1, said apparatus comprising at least a reactor containing enzymes, wherein said enzymes include a monooxygenase, and at least one of an azoreductase a tryptophanase and a tryptophan halogenase.

22. (canceled)

23. A microbial flavin-containing monooxygenase (mFMO) having sequence:
(SEQ. ID NO. 7)
MATRIAILGAGPSGLAQLRAFQAAQEKGAEIPELVCFEKQADWGGQWNYT
WRTGLDENGEPVHSSMYRYLWSNGPKEILEFADYTFDEHFGKPIASYPPR
EVLWDYIKGRVEKAGVRKYIRFNTAVRHVEFNEDSQTFTVTVQDHTTDTI
YSEEFDYVVCCTGHFSTPYVPEFEGFEKFGGRILHAHDFRDALEFKDKTV
LLVGSSYSAEDIGSQCYKYGAKKLISCYRTAPMGYKWPENWDERPNLVRV
DTENAYFADGSSEKVDAIILCTGYIHHFPFLNDDLRLVTNNRLWPLNLYK
GVVWEDNPKFFYIGMQDQWYSFNMFDAQAWYARDVIMGRLPLPSKEEMKA
DSMAWREKELTLVTAEEMYTYQGDYIQNLIDMTDYPSFDIPATNKTFLEW
KHHKKENIMTFRDHSYRSLMTGTMAPKHHTPWIDALDDSLEAYLSDKSEI
PVAKEA.

24. The process according to claim 2, wherein said step a), b) and said step of converting tryptophan or a tryptophan derivative in the presence of at least a tryptophanase, to obtain said indole or said indole derivative are carried out as a one-pot process.

25. The process according to claim 3, wherein said step a), b), said step of converting tryptophan or a tryptophan derivative in the presence of at least a tryptophanase, to obtain said indole or said indole derivative, and said step of halogenating tryptophan in the presence of at least a tryptophan halogenase and a halogen source, to obtain said halogenated derivative of tryptophan are carried out as a one-pot process.

26. The process according to claim 1, wherein said oxidizing enzyme or said reducing enzyme is coupled to a cofactor-regenerating enzyme, wherein said cofactor-regenerating enzyme is selected from glucose dehydrogenase (GDH), phosphite dehydrogenase (PTDH), formate dehydrogenase (FDH), and mixtures thereof.

27. The process according to claim 1, wherein said oxidizing enzyme is the fusion enzyme PTDH-mFMO or said reducing enzyme is PTDH-AzoA.
